(19) United States
(12) Patent Application Publication
(10) Pub. No.: US 2026/0216121 A1
(43) Pub. Date: Jul. 30, 2026

(54) 3AH-CYCLOPENTA[B]BENZOFURAN-3A-YL AS RAS INHIBITORS

(71) Applicant: SJP BIOTEC GMBH, Mellen (CH)

(72) Inventor: Krishnaraj Rajalingam, Nieder-Olm (DE)

(21) Appl. No.: 19/159,062

(22) PCT Filed: Feb. 21, 2024
(86) PCT No.: PCT/EP2024/054423

§ 371 Date: Aug. 22, 2025

(30) Foreign Application Priority Data
Feb. 23, 2023 (EP) 23158214.9

(51) Int. Cl. A61K 31/343; C07D 307/93

(52) U.S. Cl. CPC A61K 31/343; C07D 307/93

(57) ABSTRACT

The present invention relates to novel compounds and their use as a medicament, in particular for use in treating proliferative disorders. The present invention relates further to a pharmaceutical composition comprising the novel compounds. Moreover, the present invention relates to a method of inhibiting proliferation or metastasis of cancer cells or inducing their cell death in a subject in need thereof. In addition, the present invention relates to a method of inhibiting proliferation of a cell population sensitive towards inhibiting RAS, in particular KRAS, HRAS and NRAS, activation in vitro. Furthermore, the present invention relates to a method of inhibiting proliferation of a cell population sensitive towards inhibiting eIF4A complex or engaging PHB 1/2 complex in the plasma membrane in vitro. Furthermore, the present invention relates to a kit containing a formulation comprising a pharmaceutical composition comprising a compound according to the invention.

The present invention relates to novel compounds and their use as a medicament, in particular for use in treating proliferative disorders. The present invention relates further to a pharmaceutical composition comprising the novel compounds. Moreover, the present invention relates to a method of inhibiting proliferation or metastasis of cancer cells or inducing their cell death in a subject in need thereof. In addition, the present invention relates to a method of inhibiting proliferation of a cell population sensitive towards inhibiting RAS, in particular KRAS, HRAS and NRAS, activation in vitro. Furthermore, the present invention relates to a method of inhibiting proliferation of a cell population sensitive towards inhibiting eIF4A complex or engaging PHB1/2 complex in the plasma membrane or cytosol in vitro. Furthermore, the present invention relates to a kit containing a formulation comprising a pharmaceutical composition comprising a compound according to the invention.

### BACKGROUND OF THE INVENTION

Several approaches are known for the treatment of tumor diseases. A first possibility is the inhibition of RAS proteins and C-myc proteins, the major oncogenes which are mutationally activated or upregulated in a large section of human cancers.

The RAS oncogenes are frequently mutated in human cancers and among the three isoforms (KRAS, HRAS and NRAS), KRAS is the most frequently mutated oncogene. It is known that some of flavaglines like rocaglamide, a class of natural anti-tumour drugs and chemical ligands of prohibitins, inhibit RAS activation in cells by uncoupling the interaction between RAS and its effectors in the plane of the plasma membrane. Although a treatment with rocaglamide inhibits RAS-activation in KRAS-mutated cell lines, there is still a demand for compounds with a better activity towards RAS oncogenes.

It is also known that various flavagline derivatives exhibit cytotoxic properties. WO 2005/113529 A2 describes cyclopenta[b]benzofuran derivatives and their utilization for the production of medicaments, especially for the prophylaxis and/or therapy of acute or chronic diseases.

WO 2010/060891 A1 describes rocaglaol derivatives and the use of these derivatives to prevent or to limit the cardiotoxicity of an anti-neoplastic agent.

WO 2012/0666002 A1 describes flavagline derivatives and their use as neuroprotective and/or cardioprotective and/or anti-tumor agents.

WO 2017/058768 A1 describes compounds having activity as inhibitors of G12C mutant KRAS proteins.

WO 2020/078975 A1 relates to inhibitors of KRAS oncogene activation, which are flavagline derivatives with the ability to target prohibitin to inhibit KRAS activation.

N. Ribeiro et al., J. Med. Chem., 2012, 55, 100064 and Thuaud et al. Med. Chem., 2011, 54, 411 relates to flavagline derivates, in particular FL3 and FL23 which are effective in inhibition of cell proliferation and enhancement of viability at lower doses compared to rocaglalol.

Furthermore, it is known that patients frequently develop resistance to RAS oncogene inhibitors, like KRAS G12 C inhibitors (Tanaka et al., Cancer Discov, 2021, PMID 33824136). In addition, the patients treated with KRAS G12 C inhibitors often develop secondary mutations in other RAS isoforms (Awad M M et al. New England J. of Med., 2021, PMID34161704).

EP 2189453 relates to rocaglaol derivatives which may prevent or limit cardiotoxicity of an antineoplastic agent.

Engelmeier D. et al., Journal of Agricultural and Food Chemistry, American Chemical Society, US, vol. 48, no. 4, 2000-04-01, pages 1400-1404, relates to a study of plant protection from rice blast fungus. It has been shown that rocaglaol and derivates may exhibit antifungal activity.

Dumontet V. et al., Tetrahedron, Elsevier Sience Publishers, Amsterdam, NL, vol. 52, no. 20, 1996-05-13, pages 6931-6942, relates to a study of the structures of rocaglaol derivatives.

As mentioned before, there are already some approaches to inhibit RAS oncogenes and C-Myc oncogenes. However, effective targeting of this gene with small molecules is still a challenge.

Another option for the treatment of tumor diseases is the inhibition of dysregulation of protein translation. In other words, there are some developments targeting eukaryotic initiation factor (eIF4A complex) that integrates multiple oncogenic signalling inputs to the translation apparatus which includes downstream targets like C-Myc and Cyclin D1 which drives a large proportion of tumours.

Eukaryotic initiation factor 4A (eIF4A) is a DEAD-box protein containing ATPase and ATP-dependent RNA helicase required to melt local secondary structure and facilitate access of the ribosome to the mRNA template. The factor regulates the cap-dependent protein synthesis.

In mammals, there are three isoforms of eIF4A (eIF4AI, II and III), wherein eIF4AII and eIF4AIII share ~90% and ~65% identity, respectively, with the most abundant cellular factor eIF4AI. AII isoforms are DEAD-box RNA helicase family members but only the paralogs eIF4AI and eIF4AII are found in the eIF4F complex and participate in translation initiation.

WO 2017/091585 describes compounds having activity as inhibitors of eIF4A. However, the disclosed compounds have a different structure compared to the compounds according to the present invention.

Prohibitins are evolutionarily conserved proteins and recent studies revealed a critical role for prohibitins in the activation of RAS by enabling RAS-effector interaction in the plane of the plasma membrane. Polier et al, Chemistry and Biology, 19,1093-1104, 2012 showed that rocaglamides target this interaction (PHB1-CRAF) to inhibit RAS-CRAF interaction. These are several follow up studies confirming these effects.

Ernst et al, J. Med. Chem. 2020, 63, 5879 describes that the flavagline compounds rocaglamide A and Zotatifin show inhibition properties towards protein synthesis by stabilizing a translation-incompetent complex for selecting messenger RNAs (mRNAs) with eIF4A.

Accordingly, while advances have been made in this field there remains a significant need for compounds that specifically inhibit RAS and eFI4A activity, particularly with regard to eIF4A's role in regulation of cancer pathways (particularly driven by c-myc), as well as for associated compositions and methods.

In contrast to the inhibition of RAS oncogenes, which takes place at the plasma membrane possibly by engaging prohibitins driven nanoclusters among others (H. Yurugi er. Al, Journal of Cell Science, 133, 1, 2020), the inhibition of the eukaryotic factor 4A occurs in the cytosol. Therefore, two different oncogenic protein complexes are possibly targeted for profound inhibition of the tumour cell proliferation and survival. Further the activated RAS can also influence of the functioning of the eIF4A complexes through one of the effector pathways (MAPK cascades). Further most recent studies revealed that PHBs (Prohibitin) are directly associated with the eukaryotic initiation factor (elF)4F translation complex in the cytosol in CLL cells (Largeot et al., Blood, 29 (114) 2023).

As mentioned before, there are already some approaches to inhibit RAS oncogenes or to inhibit eFI4A activity. However, effective targeting of this genes with small molecules is still a challenge.

It is therefore the object of the present invention to provide pharmaceutically active compounds that have the capability to inhibit the activation of RAS oncogenes, in particular in cells at nanomolar concentrations with high specificity. Furthermore, it is the object of the present invention to provide pharmaceutically active compounds that have the capability to inhibit the RAS and eF14A activity, in particular in cells at nanomolar concentrations with high specificity, especially, with regard to both RAS's and eIF4A's role in regulation of cancer pathways.

This object is achieved by the compounds of formula (I).

### SUMMARY OF THE INVENTION

The invention relates to a compound of formula (I)

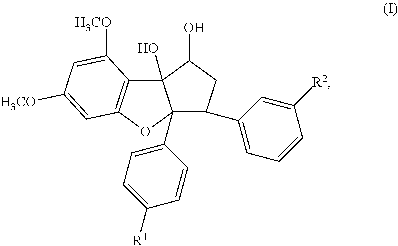

prodrugs or isotope enriched forms thereof or a pharmaceutically acceptable salt thereof, wherein

R¹ is CN,

R² is selected from hydrogen and F,

with the proviso that compounds, wherein both R¹ and R² are hydrogen are excluded.

In particular the invention relates to a compound of formula (I)

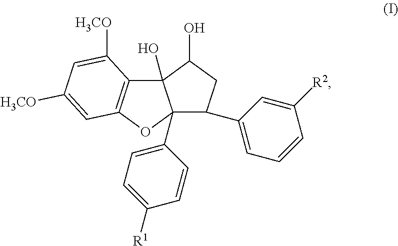

prodrugs or isotope enriched forms thereof or a pharmaceutically acceptable salt thereof,

wherein

R¹ is CN,

R² is selected from hydrogen and F.

The invention further relates to compounds of formula (I.a) or (I.b) or an enantiomeric mixture comprising the compounds of formula (I.a) and (I.b) or prodrugs or isotope enriched forms or the pharmaceutically acceptable salts thereof

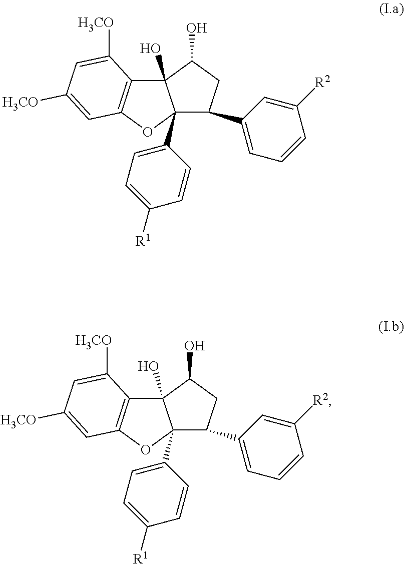

prodrugs, or isotope enriched forms thereof or a pharmaceutically acceptable salt thereof,

wherein

R¹ is CN,

R² is selected from hydrogen and F,

with the proviso that compounds, wherein both R¹ and R² are hydrogen are excluded.

In particular the invention relates to compounds of formula (I.a) or (I.b)

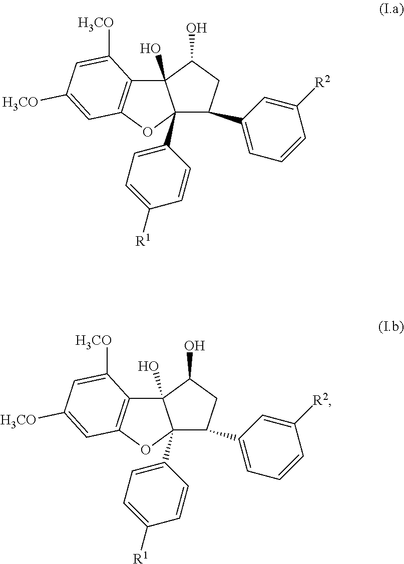

prodrugs, or isotope enriched forms thereof or a pharmaceutically acceptable salt thereof,

wherein

R¹ is CN,

R² is selected from hydrogen and F.

In particular the invention relates to an enantiomeric mixture comprising the compounds of formula (I.a) and (I.b)

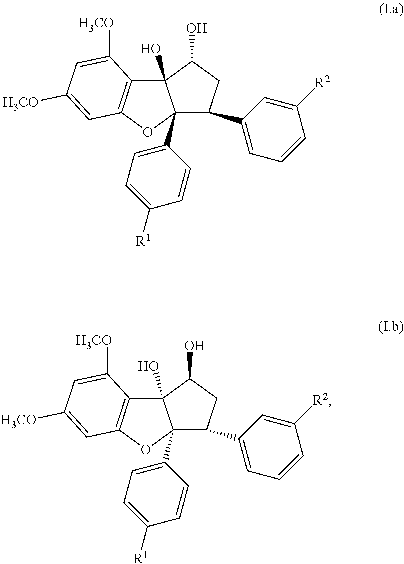

prodrugs, or isotope enriched forms thereof or a pharmaceutically acceptable salt thereof,

wherein

R¹ is CN,

R² is selected from hydrogen and F.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use as a medicament.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in the treatment and/or prophylaxis of proliferative disorders or genetic disorders or inflammatory disorders.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in the treatment and/or prophylaxis of inflammatory disorders. In particular the inflammatory disorders are selected from endometriosis and adenomyosis, in particular where RAS, preferably KRAS, is mutated.

The compounds of formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined above or below, for use in the treatment and/or prophylaxis of proliferative disorders or genetic disorders or inflammatory disorders which involves oncogenic RAS or eIF4A complex that controls the expression of genes selected from c-myc, cyclin D1 and others.

The invention further relates to a compound of formulae (I), (I.a), (I.b), (A), (B), (C), (D), (E), (F), (F) as defined above and below, an enantiomeric mixture thereof, wherein each of the compounds can be in the form of a pharmaceutically acceptable salt or form of a prodrug, or isotope enriched forms thereof, for use in the treatment and/or prophylaxis genetic disorders, wherein RAS signaling is pathologically involved. In particular the genetic disorders are selected from RASOpathies, in particular CFC (Cranio Facial Cutaneous) and NF1 (Neurofibromatosis Type 1).

The invention further relates to a compound of formulae (I), (I.a), (I.b), (A), (B), (C), (D), (E), (F), (F) as defined above and below, an enantiomeric mixture thereof, wherein each of the compounds can be in the form of a pharmaceutically acceptable salt or form of a prodrug, or isotope enriched forms thereof, for use in the treatment and/or prophylaxis genetic disorders, wherein Myc signaling is pathologically involved. In particular the genetic disorders are selected from RASOpathies, in particular CFC (Cranio Facial Cutaneous) and NF1 (Neurofibromatosis Type 1).

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in the treatment of cancer.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use as inhibitor of RAS protein activation.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in the treatment or prevention of any diseases or conditions that are associated with the activity of RAS protein (RAS oncogene).

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein RAS-signaling is involved, preferably wherein KRAS G12V, NRAS G12V, HRAS G12V, KRAS G12C, KRAS G12D, KRAS G12C/Y96D, KRAS G13C, KRAS G13D, KRASG13S, KRAS Q61H, KRAS Q61 R or KRAS Q61 K or wherein any activating mutation in KRAS, HRAS and NRAS is involved or wherein any mutation that acquires resistance to RAS inhibitors.

In particular the invention relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein RAS-signaling is involved.

In particular the invention relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein KRAS G12V, NRAS G12V, HRAS G12V, KRAS G12C, KRAS G12D, KRAS G12C/Y96D, KRAS G13C, KRAS G13D, KRASG13S, KRAS Q61H, KRAS Q61R or KRAS Q61 K is involved.

In particular the invention relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein any activating mutation in KRAS, HRAS and NRAS is involved

In particular the invention relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein any mutation that acquires resistance to RAS inhibitors.

In particular the invention relates to a compounds formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined above and below, for use as ligand of eIF4A complex or a ligand of Prohibitin or inhibitor of eIF4A complex.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use as inhibitor of eukaryotic initiation factor 4A (eIF4A).

In particular the invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use as inhibitor of eukaryotic initiation factor 4A (eIF4A), which controls expression of cancer driving genes, especially which controls expression c-myc and cyclin D1.

In particular, the invention relates to a compound of formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined above or below, for use in treating proliferative disorders, wherein eIF4A-Myc-signaling is involved.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use as a ligand of Prohibitins (PHB1/2 complex) in the plasma membrane or cytosol.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein dysregulation of elF4A is involved, preferably wherein EIF4A1, EIF4A2 or EIF4A3 or EIF4F complex is involved.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein overexpression of Prohibitins (PHB/2) is involved.

The invention further relates to a compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof, for use in treating proliferative disorders, wherein c-myc is overexpressed and/or wherein and cyclin D1 is overexpressed.

The invention further relates to a pharmaceutical composition, comprising a pharmaceutical effective amount of at least one compound selected from compounds of formulae (I), (I.a), (I.b), (A), (B), an enantiomeric mixture comprising the compounds of formula (I.a) and (I.b) or prodrugs or isotope enriched forms or a pharmaceutically acceptable salt thereof, as defined above and below, and one or more pharmaceutical acceptable carrier.

The invention further relates to a pharmaceutical composition, comprising a pharmaceutical effective amount of at least one compound selected from compounds of formulae ((I), (I.a), (I.b), (A), (B), an enantiomeric mixture comprising the compounds of formula (I.a) and (I.b) or prodrugs or a pharmaceutically acceptable salt thereof, for use in the prophylaxis and/or treatment of proliferative disorders or genetic disorders.

The invention further relates to a method of inhibiting growth, proliferation, or metastasis of cancer cells in a subject in need thereof, said method comprising administering to the subject a therapeutically effective amount of at least one compound of formulae (I), (I.a), (I.b), (A), (B) as defined above and below, an enantiomeric mixture thereof, wherein each of the compounds can be in the form of a pharmaceutically acceptable salt or in form of a prodrug, or a pharmaceutical composition as defined above or below.

The invention further relates to a method of inhibiting proliferation of a cell population sensitive towards inhibiting RAS activation, in particular inhibiting KRAS, HRAS or NRAS activation in vitro or ex vivo, the method comprising contacting the cell population with at least one compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof or a pharmaceutical composition as defined above and below.

The invention further relates to method of inhibiting proliferation of a cell population sensitive towards inhibiting elF4A or its downstream targets, in particular c-myc and/or cyclin D1 in vitro or ex vivo, the method comprising contacting the cell population with at least one compound of formulae (I), (I.a), (I.b), (A), (B) as defined above and below, an enantiomeric mixture thereof, wherein each of the compounds can be in the form of a pharmaceutically acceptable salt or in form of a prodrug or isotope enriched forms thereof or a pharmaceutical composition as defined above and below.

The invention further relates to a kit comprising formulation comprising: a1) at least one compound of formulae (I), (I.a), (I.b), (A), (B), as defined above and below, an enantiomeric mixture thereof, wherein each of the compounds can be in the form of a pharmaceutically acceptable salt or in form of a prodrug or isotope enriched forms thereof or a2) a pharmaceutical composition comprising at least one compound of formulae (I), (I.a), (I.b), (A), (B) as defined above and below, an enantiomeric mixture thereof, wherein each of the compounds can be in the form of a pharmaceutically acceptable or in form of a prodrug, or a prodrug or isotope enriched forms thereof or a therapeutically acceptable salt thereof and a pharmaceutically acceptable carrier; and b) instructions for dosing of the pharmaceutical composition for the treatment of a disorder in which inhibition of RAS activation is effective in treating the disorder.

The invention further relates to a kit comprising formulation comprising: a1) at least one compound of formulae (I), (I.a), (I.b), (A), (B), as defined above and below, an enantiomeric mixture thereof, wherein each of the compounds can be in the form of a pharmaceutically acceptable salt or in form of a prodrugs or isotope enriched forms or a2) a pharmaceutical composition comprising at least one compound of formulae (I), (I.a), (I.b), (A), (B) as defined above and below, an enantiomeric mixture thereof, wherein each of the compounds can be in the form of a pharmaceutically acceptable or in form of a prodrug, or prodrug or isotope enriched forms thereof or a therapeutically acceptable salt thereof and a pharmaceutically acceptable carrier; and b) instructions for dosing of the pharmaceutical composition for the treatment of a disorder in which inhibition of dysregulation of protein translation, wherein eIF4A is involved, is effective in treating the disorder.

The invention further relates to a kit containing a formulation comprising: a1) at least one compound selected from compounds of formulae (I), (I.a), (I.b), an enantiomeric mixture comprising the compounds of formula (I.a) and (I.b) or prodrugs or isotope enriched forms thereof or a pharmaceutically acceptable salt thereof, as defined above or below, or a2) a pharmaceutical composition as defined above or below; and b) instructions for dosing of the pharmaceutical composition for the treatment of a disorder in which inhibition of RAS activation or the downstream signaling pathways, in particular including C-Myc upregulation and/cyclin D1 upregulation is effective in treating the disorder.

The invention has the following advantages:
The compounds according to the invention exhibit advantageous RAS inhibition properties. In other words the compounds according to the invention qualify as inhibitors of RAS oncogene activation by inhibiting the prohibitin pathway, in particular inhibiting EGF-induced RAS-GTP loading in cells which is measured by the ability of RAS to bind to its effector proteins like RAF kinases.
The compounds according to the invention prevent the activation of RAS as the interaction between RAS and its effectors is uncoupled due to defects in nanoclustering of RAS in the plane of the plasma membrane.
It is possible to identify potential candidate substances, by conducting a screening of compounds according to the invention that are able to inhibit the activation of RAS by directly disrupting the interaction between activated RAS (both by EGF and mutational activation) and the RAS binding domain (RBD) of the CRAF kinase in cells or other RAS interaction domains like the RA (RAS associating) domain.
The compounds are obtainable on reasonable scale for further development.
The compounds are soluble with good pharmacokinetic/pharmacodynamic (PK/PD) properties. These compounds inhibit RAS irrespective of the mutations at nanomolar range and e.g. exhibit inhibitory effects on KRAS, NRAS and HRAS.
In addition, the compounds, in particular at increasing concentrations and at longer time points post treatment, inhibit eIF4A complex, which is responsible for the translation of several oncogenes like c-myc and cyclin D1. This complex has been targeted by the so called STIs (Selective translational inhibitors).
A dual luciferase assay has been established to measure eIF4A activity in vitro and the compounds are further screened for the inhibition of eIF4A.

### DESCRIPTION OF THE INVENTION

### Compounds of Formula (I)

Unless specifically stated otherwise herein, references made in the singular may also include the plural. For example, “a” and “an” may refer to either one, or one or more.

The compounds of formulae (I), (I.a), (I.b), (A), (B), an enantiomeric mixture thereof, may form salts which are also within the scope of this invention. The term “salt(s)” as employed herein, denotes acidic and/or basic salts formed with inorganic and/or organic acids and bases. Pharmaceutically acceptable (i.e. non-toxic, physiologically acceptable) salts are preferred, although other salts are also useful, e.g., in isolation or purification steps which may be employed during preparation. Salts of the compounds of formulae (I), (I.a), (I.b), (A), (B), or an enantiomeric mixture thereof may be formed, for example, by reacting a compound of formulae (I), (I.b), (A), (B), or an enantiomeric mixture thereof with at least one acid or base. The acid or base is added in an amount suitable for partial or complete neutralization, e.g. an equivalent amount.

The phrase “pharmaceutically acceptable salt(s)” as used herein, unless otherwise indicated, includes salts containing pharmacologically acceptable anions or cations, such as chloride, bromide, iodide, nitrate, sulfate, bisulfate, phosphate, hydrogen phosphate, dihydrogen phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, sulfate, benzenesulfonate, p-toluenesulfonate and palmoate [i.e. 4,4′-methylene-bis-(3-hydroxy-2-naphthoate)] salts.

Any compound according to the invention can be prepared and used in prodrug form. A suitable prodrug has chemically or metabolically cleavable group(s) and becomes, by solvolysis or under physiological conditions, a compound that is pharmaceutically active in vivo. A prodrug can be formed in a conventional manner by reaction of a functional group of the compound (such as an amino, hydroxy or carboxy group). Prodrugs often offer advantages of better metabolism, potency, solubility, tissue compatibility, or delayed release in mammals.

The term “prodrug” refers to a metabolic precursor of a compound of the invention that is pharmaceutically acceptable. A prodrug may be inactive when administered to a subject in need thereof but is converted in vivo to an active compound of the invention. Prodrugs are typically rapidly transformed in vivo to yield the parent compound of the invention, for example, by hydrolysis in blood.

In other words, in the terms of the present invention, “prodrugs” are compounds which are metabolized in vivo to give the compounds of the invention of formula I. Typical examples for prodrugs are for example described in C. G. Wermeth (editor): The Practice of Medicinal Chemistry, Academic Press, San Diego, 1996, pages 671-715 as well as J. Rautio et al., NATURE REVIEWS, Drug Discovery, Vol. 17, 2018, p. 559. Examples are phosphates, carbonates, carbamates, aminoacids, esters (carboxylic acid esters), ethers, amides, peptides, ureas and the like. In the present case, suitable prodrugs can be compounds of formula I wherein the hydroxy group is bonded to any group that, when the prodrug of the compound of the invention is administered to a mammalian subject, cleaves to form a free hydroxy group.

Myc is a family of regulator genes and proto-oncogenes that code for transcription factors. The Myc family consists of three related human genes: c-myc (MYC), I-myc (MYCL), and n-myc (MYCN). c-myc (also sometimes referred to as MYC) was the first gene to be discovered in this family, due to homology with the viral gene v-myc. In cancer, c-myc is often constitutively (persistently) expressed. This leads to the increased expression of many genes, some of which are involved in cell proliferation, contributing to the formation of cancer.

Cyclin D1 is a protein that in humans is encoded by the CCND1 gene. The CCND1 gene encodes the cyclin D1 protein. The human CCND1 gene is located on the long arm of chromosome 11 (band 11q13). It is 13,388 base pairs long, and translates into 295 amino acids. Cyclin D1 is expressed in all adult human tissues with the exception of cells derived from bone marrow stem cell lines (both lymphoid and myeloid).

In the context of the present invention, a chemical structure that does not explicitly show a specific stereochemical orientation usually means all possible stereoisomers and mixtures thereof, unless indicated otherwise, for example,

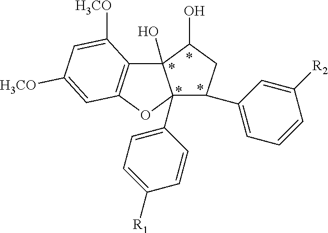

in which * designates the asymmetry centers.

“Chiral compounds” in the sense of the invention are compounds that contain no improper axis of rotation (Sₙ). In the context of the present invention, they are in particular compounds with at least four chirality centers and without Sₙ-symmetry.

“Stereoisomers” in the context of the invention are compounds of identical constitution but different atomic arrangement in the three-dimensional space.

“Enantiomers” are stereoisomers which behave like image to mirror image to one another, e.g. compounds of formulae (I.a) and (I.b) are enantiomers. The “enantiomeric excess” (ee) achieved during asymmetric synthesis is given here by the following formula: ee [%]=(R−S)/(R+S)×100. R and S are the descriptors of the CIP system for the two enantiomers and describe the absolute configuration on the asymmetric atom. The enantiomerically pure compound (ee=100%) is also referred to as “homochiral compound”.

“Diastereomers” are stereoisomers which are not enantiomeric to one another.

The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

Compounds of the invention or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography.

Relative configuration in stereochemistry (relative stereochemistry) is the arrangement of atoms or groups of atoms that is described relative to other atoms or groups of atoms in the molecule. In other words, this term describes the position of atoms or groups of atoms in space in relation to other atoms or groups of atoms that are located elsewhere in the molecule.

Absolute configuration in stereochemistry (absolute stereochemistry) is the arrangement of atoms or group of atoms that is described independently of any other atom or group of atoms in the molecule. This type of configuration is defined for chiral molecular entities and their stereochemical descriptions (e.g. R or S).

Syn means that with regard to the orientation of the substituents on the 5-membered ring they are bound to (4 asymmetric carbon atoms) all substituents point in the same direction relative to the plane of the 5-membered ring.

Racemic mixture or racemate is defined as a mixture of compounds consisting of two molecules structured like image and mirror image (=enantiomers) and which are present in an equimolar mixture, i.e. in the ratio 1:1 (50:50).

Further, the compounds of formula (I)

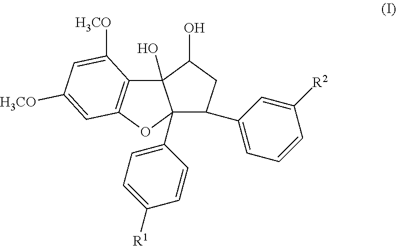

in which * designates the asymmetry centers, represent the isomers of formulae (I.a), (I.b), (I.c), (I.d), (I.e), (I.f), (I.g), (I.h), (I.i), (I.j), (I.k), (I.l), (I.m), (I.n), (I.o) and (I.p):

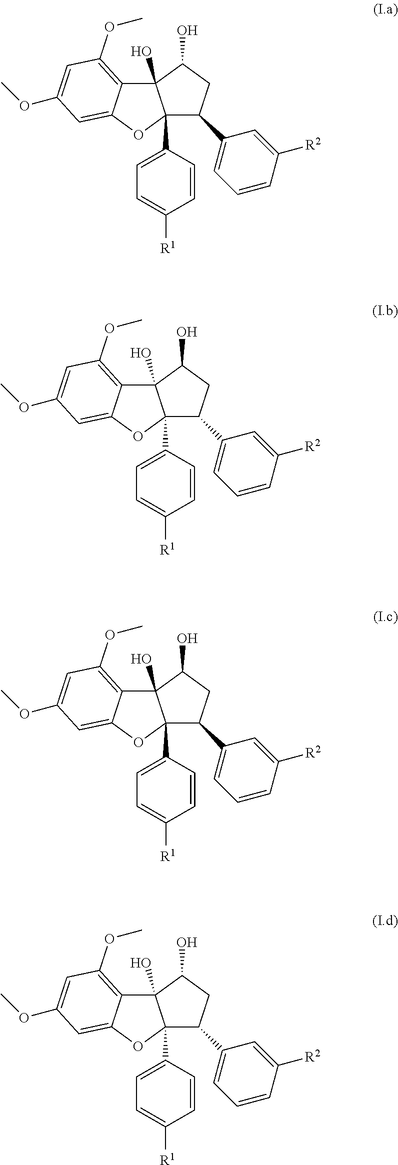

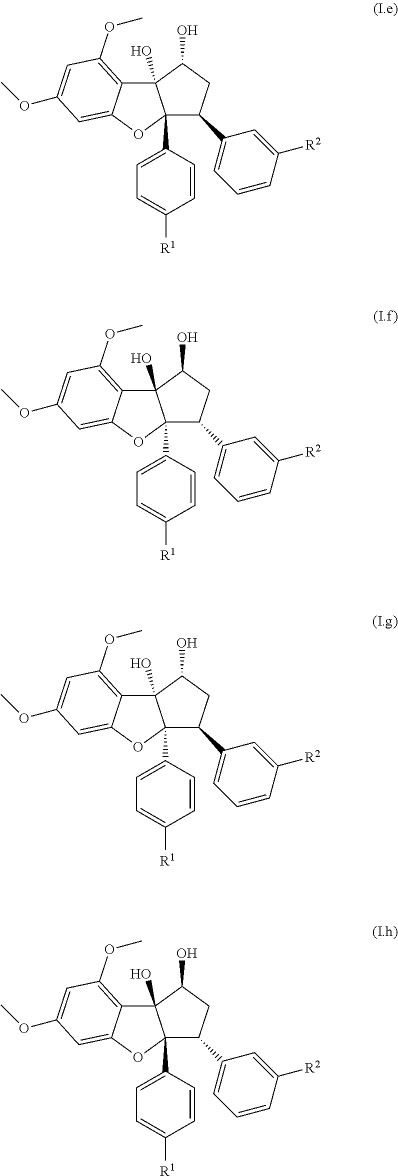

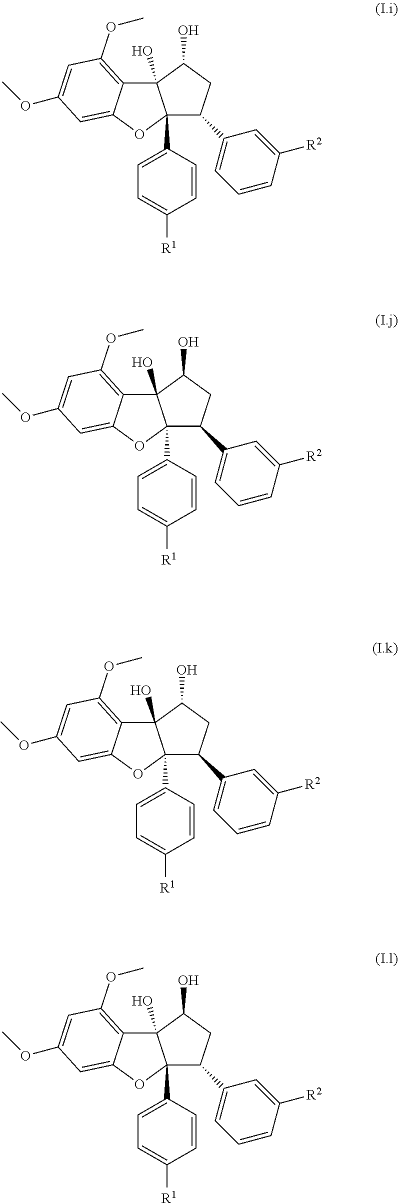

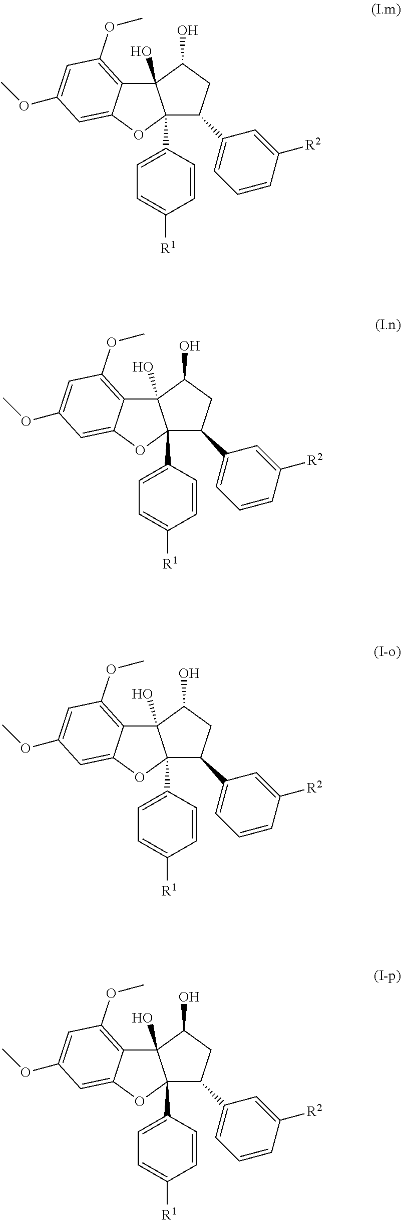

wherein, R¹ and R² have one of the meanings as defined above or below.

The compounds of formulae (I.a) to (I.p) are specified by their absolute stereochemistry.

In a preferred embodiment the compound of formula (I) is a mixture of at least two enantiomers (I.a) to (I.p) or a mixture of the prodrugs or isotope enriched forms thereof, or a mixture of the pharmaceutically acceptable salt thereof, wherein one enantiomer is enriched.

Preferably the compound of formula (I) is a mixture of (I.a) and (I.b) or a mixture of the pharmaceutically acceptable salt thereof, wherein the enantiomer excess (ee) of the enantiomer of formula (I.a) is at least 20%, preferably at least 50%, in particular at least 80%, especially at least 99%.

Preferred are compound of formula (I) according to the invention, or a prodrug or isotope enriched forms thereof or a pharmaceutically acceptable salt thereof, wherein R² is hydrogen.

Further, preferred are compound of formula (I) according to the invention, or a progrug or a pharmaceutically acceptable salt thereof, wherein R² is F.

Another special embodiment are the compounds of formula (I), wherein R¹ and R² are selected from the definition given in one line of table 1:

TABLE 1

| Nr. | R¹ | R² |
|---|---|---|
| 1. | CN | F |
| 2. | CN | hydrogen |

Another special embodiment are the compounds selected from A, B and the mixture each of the of compounds A to B with its respective enantiomer:

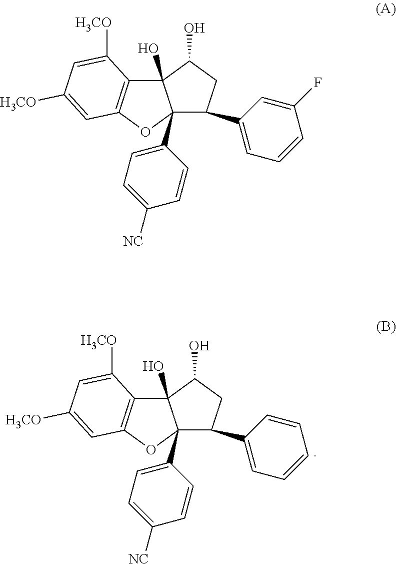 [SMILES: COc1cc(OC)c2c(c1)O[C@@]1(c3ccc(C#N)cc3)[C@H](c3cccc(F)c3)C[C@@H](O)[C@@]21O ; COc1cc(OC)c2c(c1)O[C@@]1(c3ccc(C#N)cc3)[C@H](c3ccccc3)C[C@@H](O)[C@@]21O]

Preferred is a compound of formula (A) or an enantiomeric mixture comprising the compounds of formula (A) and its enantiomer, in particular wherein the enantiomer excess (ee) of the enantiomer of formula (A) is at least 20%, preferably at least 50%, in particular at least 80%, especially at least 99%.

Preferred is a compound of formula (B) or an enantiomeric mixture comprising the compounds of formula (B) and its enantiomer, in particular wherein the enantiomer excess (ee) of the enantiomer of formula (B) is at least 20%, preferably at least 50%, in particular at least 80%, especially at least 99%.

Especially preferred is compound A.

Especially preferred is compound B.

The compounds of the present invention can be synthesized using the methods known in the prior art and together with methods known from synthetic organic chemistry, or variations thereof as appreciated by those skilled in the art.

### Pharmaceutical Composition

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “therapeutically effective” is intended to qualify the amount of each agent, which will achieve the goal of improvement in disorder severity and the frequency of incidence, while avoiding adverse side-effects typically associated with alternative therapies. For example, effective anticancer agents prolong the survivability of the patient or his/her life quality, inhibit the rapidly proliferating cell growth associated with the neoplasm, or effect a regression of the neoplasm.

The terms “treat,” “treating,” and “treatment,” as used herein, refer to any type of intervention or process performed on, or administering an active agent to, the subject with the objective of reversing, alleviating, ameliorating, inhibiting or slowing down or preventing the progression, development, severity or recurrence of a symptom, complication, condition or biochemical indicia associated with a disease. By contrast, “prophylaxis” or “prevention” refers to administration to a subject who does not have a disease to prevent the disease from occurring.

As used herein, the term “cell” is meant to refer to a cell that is in vitro, ex vivo or in vivo. In the sense of the invention, an ex vivo cell can be part of a tissue sample excised from an organism such as a mammal. In the sense of the invention, an in vitro cell can be a cell in a cell culture. In the sense of the invention, an in vivo cell is a cell living in an organism such as a mammal.

The term “patient” includes humans and animals that receive either therapeutic or prophylactic treatment.

The term “subject” includes any human or animal. For example, the methods and compositions herein disclosed can be used to treat a subject having cancer.

A (non-human) animal includes all vertebrates, e.g. mammals and non-mammals, including cows, sheep, pigs, goats, horses, poultry, dogs, cats, non-human primates, rodents etc. In one embodiment, the subject is a human subject.

The phrase “pharmaceutically acceptable carrier” as used herein means a pharmaceutically acceptable material, composition or vehicle, such as a liquid or solid diluent, solvent, excipient, manufacturing aid (e.g. lubricant) or encapsulating material, involved in carrying or transporting the subject compound from one organ, or portion of the body, to another organ, or portion of the body. Each carrier must be “acceptable” in the sense of being compatible with the other ingredients of the formulation.

Suitable other ingredients are the afore-mentioned carrier and further additives, including adjuvants, preserving agents, fillers, flow regulating agents, disintegrating agents, wetting agents, emulsifying agents, suspending agents, sweetening agents, flavoring agents, bittering agents, perfuming agents, antibacterial agents, antifungal agents, lubricating agents, dispensing agents, etc. Suitable additives are selected depending on the nature of the mode of administration and dosage forms; and not injurious to the patient.

The term “pharmaceutical composition” means a composition comprising a compound of the invention in combination with at least one further compound selected from
a) at least one further pharmaceutically active substance and
b) at least one additional pharmaceutically acceptable carrier and or additive.

### Ras

The term “RAS inhibitor” refers to an agent capable of decreasing RAS protein levels, decreasing RAS activity levels and/or inhibiting RAS expression levels in the cells. The RAS inhibitor may be a reversible or irreversible inhibitor. As used herein, “RAS” protein refers to a protein that is a member of a family of related proteins that are expressed in all human and animal cell lineages and organs. AII RAS protein family members belong to a class of proteins called small GTPase (also known as small G proteins, a family of hydrolase enzymes that can bind and hydrolyse GTP), and are involved in transmitting signals within cells (cellular signal transduction). RAS is the prototypical member of the RAS superfamily of proteins, which are all related in three-dimensional structure and regulate diverse cell behaviours. When RAS is ‘switched on’ by incoming signals, it subsequently switches on other proteins, which ultimately turn on genes involved in cell growth, differentiation, and survival. Mutations in RAS genes can lead to the production of permanently activated RAS proteins, which can cause unintended and overactive signaling inside the cell, even in the absence of incoming signals. Because these signals result in cell growth and division, overactive RAS signaling can ultimately lead to cancer. The three RAS genes in humans (HRAS, KRAS, and NRAS) are the most common oncogenes in human cancer. As mentioned, the clinically most notable members of the RAS subfamily are HRAS, KRAS and NRAS. However, there are other members of this subfamily, which are e.g. selected from DIRASI, DIRAS2, DIRAS3, ERAS, GEM, MRAS, NKIRAS1, NKIRAS2, NRAS, RALA, RALB, RAP1A, RAP1B, RAP2A, RAP2B, RAP2C, RASD1, RASD2, RASL10A, RASLIOB, RASL11A, RASL11B, RASL12, REM1, REM2, RERG, RERGL, RRAD, RRAS, RRAS2.

The compound(s) of formulae (I), (I.a), (I.b), (A), (B) and an enantiomeric mixture as defined above and a pharmaceutical composition comprising in at least one compound of formulae (I), (I.a), (I.b), (A), (B) and an enantiomeric mixture as defined above may be administered to humans and animals, preferably humans.

In principle any method of administration may be used to deliver the compound or pharmaceutical composition according to the invention to a subject. Suitable methods of administration are orally, enterally, parenterally, intravenously, topically, intramuscular, subcutaneous/dermal routes.

The compound(s) of formulae (I), (I.a), (I.b), (A), (B) and an enantiomeric mixture as defined above can selectively decrease RAS protein levels, decrease RAS activity levels and/or inhibit RAS expression levels in the cells. For example, the compound(s) of formulae (I), (I.a), (I.b), (A), (B) and an enantiomeric mixture as defined above can be used to selectively decrease RAS activity levels and/or inhibit RAS expression levels in cells or in an individual in need of a decrease in RAS protein levels, decrease in RAS activity levels and/or inhibition of RAS expression levels by administering an inhibiting amount of compound(s) of formulae (I), (I.a), (I.b), (A), (B) and an enantiomeric mixture as defined above or a salt thereof.

In one embodiment, the present invention provides a combined preparation of compound(s) of formulae (I), (I.a), (I.b), (A), (B) and an enantiomeric mixture as defined above, and/or a pharmaceutically acceptable salt thereof and/or prodrugs or isotope enriched forms thereof, and an additional therapeutic agent(s) for simultaneous, separate or sequential use in the treatment and/or prophylaxis of multiple diseases, preferably of proliferative disorders (e.g. cancer), in particular disorders associated with the activity of RAS protein.

Additional therapeutic agent(s) are selected from chemotherapeutic agents, radiotherapeutic agents, immuno-oncology agents, and combinations thereof.

In one aspect, the compound(s) of formulae (I), (I.a), (I.b), (A), (B) and an enantiomeric mixture as defined above are sequentially administered prior to administration of the immuno-oncology agent. In another aspect, compound(s) of formulae (I), (I.a), (I.b), (A), (B) and an enantiomeric mixture as defined above are administered concurrently with the immuno-oncology agent. In yet another aspect, compound(s) of formulae (I), (I.a), (I.b), (A), (B) and an enantiomeric mixture as defined above are sequentially administered after administration of the immuno-oncology agent.

In another aspect, compound(s) of formulae (I), (I.a), (I.b), (A), (B) and an enantiomeric mixture as defined above may be co-formulated with an immuno-oncology agent.

Immuno-oncology agents include, for example, a small molecule drug, antibody or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Suitable of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1 BBL, CD137 (4-1BB), TRAIL/Apo2-L, TRAILRI/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTpR, LIGHT, DcR3, HVEM, VEGETLiA, TRAMP/DR3, EDAR, EDA1, XEDAR, EDA2, TNFR1, Lymphotoxin a/TNFp, TNFR2, TNFa, LTpR, Lymphotoxin a 1b2, FAS, FASL, RELT, DR6, TROY, NGFR.

In one aspect, T cell responses can be stimulated by a combination of compound(s) of formulae (I), (I.a), (I.b), (A), (B) and an enantiomeric mixture as defined above and one or more of
(i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and
(ii) an agonist of a protein that stimulates T cell activation such as B7-1, B7-2, CD28, 4-1BB (CD 137), 4-1 BBL, ICOS, ICOS-L, 0X40, OX40L, GITR, GITRL, CD70, CD27, CD40, DR3 and CD28H.

Other agents that can be combined with compound(s) of formulae (I), (I.a), (I.b), (A), (B) and an enantiomeric mixture as defined above for the treatment of cancer include antagonists of inhibitory receptors on NK cells or agonists of activating receptors on NK cells. For example, compound(s) of formulae ((I), (I.a), (I.b), (A), (B) and an enantiomeric mixture as defined above can be combined with antagonists of KIR, such as Lirilumab.

Yet other agents for combination therapies include agents that inhibit or deplete macrophages or monocytes, including but not limited to CSF-1 R antagonists such as CSF-1 R antagonist antibodies including RG7155.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy can also embrace the administration of the therapeutic agents as described above in further combination with other biologically active ingredients and non-drug therapies (e.g surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Types of cancers that may be treated with the compound of formulae (I), (I.a), (I.b), (A), (B) and an enantiomeric mixture as defined above include, but are not limited to, prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, haematological malignancies (including blood, bone marrow and lymph nodes) or testicular seminoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compound(s) of formulae (I), (I.a), (I.b), (A), (B) and an enantiomeric mixture as defined above for treatment of RAS protein associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compound(s) of formulae (I), (I.a), (I.b), (A), (B) and an enantiomeric mixture as defined include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compound(s) of formulae (I), (I.a), (I.b), (A), (B) and an enantiomeric mixture as defined above may also be combined with immunotherapy drugs, including cytokines such as interferon alpha, interleukin 2, and tumor necrosis factor (TNF) in the treatment of melanoma. Compound(s) of formulae (I), (I.a), (I.b), (A), (B) and an enantiomeric mixture as defined above may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compound(s) of formulae (I), (I.a), (I.b), (A), (B) and an enantiomeric mixture as defined, using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-α), etoposide, and teniposide.

Other cytotoxic agents include navelbene, CPT-11, anastrazole, letrazole, capecitabine, reloxafme, and droloxafme.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1 BB and PD-1, or antibodies to cytokines (IL-1O or TGF-b).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

In a specific embodiment of the present invention, at least one compound of (I), (I.a), (I.b), (A), (B) and an enantiomeric mixture as defined above and at least one chemotherapeutic agent are administered to the patient concurrently or sequentially. In other words, at least one compound(s) of formulae (I), (I.a), (I.b), (A), (B) and an enantiomeric mixture as defined above may be administered first, at least one chemotherapeutic agent may be administered first, or at least one compound of formulae (I), (I.a), (I.b), (A), (B) and an enantiomeric mixture as defined above may be administered at the same time. Additionally, when more than one compound of formulae (I), (I.a), (I.b), (A), (B) and an enantiomeric mixture as defined above and/or chemotherapeutic agent is used, the compounds may be administered in any order.

The invention also provides pharmaceutically compositions which comprise a therapeutically effective amount of one or more of the compound(s) of formulae (I), (I.a), (I.b), (A), (B) and an enantiomeric mixture as defined above, formulated together with one or more pharmaceutically acceptable carriers (additives) and/or diluents, and optionally, one or more additional therapeutic agents described above.

The compound(s) of formulae (I), (I.a), (I.b), (A), (B) and an enantiomeric mixture as defined above, may be administered by any suitable route, preferably in the form of a pharmaceutical composition adapted to such a route, and in a dose effective for the treatment intended. The compound(s) and compositions of the compound(s) of formulae (I), (I.a), (I.b), (A), (B) an enantiomeric mixture as defined above, can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g. as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or animal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, bittering agents, coloring agents, demulcents, antioxidants, and preserving agents.

A tablet can, for example, be prepared by admixing at least one compound of formulae (I), (I.a), (I.b), (A), (B) and an enantiomeric mixture as defined above, and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasantly tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of formulae (I), (I.a), (I.b), (A), (B) and an enantiomeric mixture as defined above, and/or at least one salt thereof with at least one inert solid diluent, such as, for example, calcium carbonate; calcium phosphate; and kaolin.

Soft gelatin capsules can, for example, be prepared by mixing at least one compound of formulae (I), (I.a), (I.b), (A), (B) and an enantiomeric mixture as defined above, and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formulae (I), (I.a), (I.b), (A), (B) and an enantiomeric mixture as defined above and/or at least one pharmaceutically acceptable salt thereof and/or prodrugs or isotope enriched forms thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, hydroxypropyl-methylcellulose and hydroxypropyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formulae (I), (I.a), (I.b), (A), (B) and an enantiomeric mixture as defined above and/or at least one pharmaceutically acceptable salt thereof and/or prodrugs or isotope enriched forms thereof in either a vegetable oil, such as, for example, arachis oil, olive oil, sesame oil and coconut oil or in mineral oil, such as, for example, liquid paraffin. An oily suspension can also contain at least one thickening agent, such as, for example, beeswax, hard paraffin and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formulae (I), (I.a), (I.b), (A), (B) and an enantiomeric mixture as defined above and/or at least one pharmaceutically acceptable salt thereof and/or prodrugs or isotope enriched forms thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of formulae (I), (I.a), (I.b), (A), (B) and an enantiomeric mixture as defined above and/or at least one pharmaceutically acceptable salt thereof and/or prodrugs or isotope enriched forms thereof can, for example, be prepared as an oil-in-water emulsion. The oily phase of the emulsions comprising compound(s) of formulae (I), (I.a), (I.b), (A), (B) and an enantiomeric mixture as defined above may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate.

Preferably, a hydrophilic emulsifier is included together with a lipophilic emulsifier which acts as a stabilizer. It is also preferred to include both an oil and a fat. Together, the emulsifier(s) with or without stabilize) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compound(s) of formulae (I), (I.a), (I.b), (A), (B) and an enantiomeric mixture as defined above and/or at least one pharmaceutically acceptable salt thereof and/or prodrugs or isotope enriched forms thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, water or with cyclodextrin solubilization (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of formulae (I), (I.a), (I.b), (A), (B) and an enantiomeric mixture as defined above in an oily phase, such as, for example, a mixture of soybean oil and lecithin; 2) combining the compound(s) of formulae (I), (I), (I.a), (I.b), (A), (B) and an enantiomeric mixture as defined above containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, AIIen, L. V. Jr. et al. Remington: The Science and Practice of Pharmacy (2 Volumes), 22nd Edition (2012), Pharmaceutical Press.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol poly ethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depend on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of formulae (I), (I.a), (I.b), (A), (B) and an enantiomeric mixture as defined above and/or at least one pharmaceutically acceptable salt thereof and/or prodrugs or isotope enriched forms thereof, and optionally an additional agent selected from any pharmaceutically acceptable carrier, adjuvant, and vehicle. Alternate compositions of this invention comprise a compound of the formulae (I), (I.a), (I.b), (A), (B) and an enantiomeric mixture as defined above, or a prodrug or isotope enriched forms thereof, and a pharmaceutically acceptable carrier, adjuvant, or vehicle.

The present invention also includes pharmaceutical kits useful, for example, in the treatment or prevention of RAS protein-associated diseases. Thus, the present invention also relates to a kit containing a formulation comprising: a) a pharmaceutical composition comprising a compound of formulae (I), (I.a), (I.b), (A), (B) and an enantiomeric mixture as defined above, or a therapeutically acceptable salt and/or prodrugs or isotope enriched forms thereof and a pharmaceutically acceptable carrier; and b) instructions for dosing of the pharmaceutical composition for the treatment of a disorder in which inhibition of RAS activation is effective in treating the disorder.

Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compound(s) of the formulae (I), (I.a), (I.b), (A), (B) and an enantiomeric mixture may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g. oral tablets, capsules, elixirs, and syrups, and consistent with conventional pharmaceutical practices. Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition. A typical capsule for oral administration contains at least one of the compound of the formulae (I), (I.a), (I.b), (A), (B) and an enantiomeric mixture (250 mg), lactose (75 mg), and magnesium stearate (15 mg). The mixture is passed through a 60 mesh sieve and packed into a no. 1 gelatin capsule.

A typical injectable preparation is produced by aseptically placing at least one of the compound of the formulae (I), (I.a), (I.b), (A), (B) and an enantiomeric mixture (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compound of formulae (I), (I.a), (I.b), (A), (B) and an enantiomeric mixture as defined above, alone or in combination with a pharmaceutical carrier. Optionally, compound(s) of formulae (I), (I.a), (I.b), (A), (B) and an enantiomeric mixture as defined above can be used alone, in combination with other compound(s) of formulae (I), (I.a), (I.b), (A), (B) and an enantiomeric mixture as defined above, or in combination with one or more other therapeutic agent(s), e.g. an anticancer agent or other pharmaceutically active material.

Regardless of the route of administration selected, the compound(s) of formulae (I), (I.a), (I.b), (A), (B) and an enantiomeric mixture as defined above, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound(s) of formulae (I), (I.a), (I.b), (A), (B) and an enantiomeric mixture as defined above employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of compound(s) of formulae (I), (I.a), (I.b), (A), (B) and an enantiomeric mixture as defined above employed in the pharmaceutical composition at levels lower than that required in order to achieve the desired therapeutic effect and gradually increase the dosage until the desired effect is achieved.

In general, a suitable daily dose of compound(s) of formulae (I), (I), (I.a), (I.b), (A), (B) and an enantiomeric mixture as defined above will be that amount of the compound which is the lowest dose effective to produce a therapeutic effect. Such an effective dose will generally depend upon the factors described above. Generally, oral, intravenous, intracerebroventricular and subcutaneous doses of the compound(s) of formulae (I), (I.a), (I.b), (A), (B) and an enantiomeric mixture as defined above for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for compound(s) of formulae (I), (I.a), (I.b), (A), (B) and an enantiomeric mixture as defined above to be administered alone, it is preferable to administer the compound as a pharmaceutical formulation (composition).

The above other therapeutic agents, when employed in combination with the compound(s) of formulae (I), (I.a), (I.b), (A), (B) and an enantiomeric mixture as defined above, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Eukaryotic Initiation Factor 4A

The mechanisms governing the basic subsistence of eukaryotic cells are immensely complex; it is therefore unsurprising that regulation occurs at a number of stages of protein synthesis. Human translational control is of increasing research interest as it has connotations in a range of diseases. Orthologs of many of the factors involved in human translation are shared by a range of eukaryotic organisms. Synthesis of protein from mature messenger RNA in eukaryotes is divided into translation initiation, elongation, and termination of these stages; the initiation of translation is the rate limiting step. Within the process of translation initiation; the bottleneck occurs shortly before the ribosome binds to the 5′ m7GTP facilitated by a number of proteins; it is at this stage that constrictions born of stress, amino acid starvation etc. take effect.

Eukaryotic initiation factor complex 2 (elF2) forms a ternary complex with GTP and the initiator Met-tRNA—this process is regulated by guanine nucleotide exchange and phosphorylation and serves as the main regulatory element of the bottleneck of gene expression. Before translation can progress to the elongation stage, a number of initiation factors must facilitate the synergy of the ribosome and the mRNA and ensure that the 5′

UTR of the mRNA is sufficiently devoid of secondary structure. Binding in this way is facilitated by group 4 eukaryotic initiation factors; eIF4F has implications in the normal regulation of translation as well as the transformation and progression of cancerous cells.

eIF4F is responsible for the binding of capped mRNA to the 40S ribosomal subunit via elF3. The mRNA cap is bound by eIF4E (25 kDa), eIF4G (185 kDa) acts as a scaffold for the complex whilst the ATP-dependent RNA helicase eIF4A (46 kDa) processes the secondary structure of the mRNA 5′ UTR to render it more conducive to ribosomal binding and subsequent translation. Together these three proteins are referred to as eIF4F. For maximal activity; eIF4A also requires eIF4B (80 kDa), which itself is enhanced by eIF4H (25 kDa). Once bound to the 5′ cap of mRNA, this 48S complex then searches for the (usually) AUG start codon and translation begins.

The compound(s) of formulae (I), (I.a), (I.b), (A), (B) and an enantiomeric mixture as defined above and a pharmaceutical composition comprising in at least one compound of formulae (I), (I.a), (I.b), (A), (B) and an enantiomeric mixture as defined above may be administered to humans and animals, preferably humans.

In principle any method of administration may be used to deliver the compound or pharmaceutical composition according to the invention to a subject. Suitable methods of administration are orally, enterally, parenterally, intravenously, topically, intramuscular, subcutaneous routes.

The compound(s) of formulae (I), (I.a), (I.b), (A), (B) and an enantiomeric mixture as defined above can selectively decrease activity of eIF4A, decrease eIF4A activity levels and/or inhibit activity of elF4 in the cytosol. For example, the compound(s) of formulae (I), (I.a), (I.b), (A), (B) and an enantiomeric mixture as defined above can be used to selectively decrease eIF4A activity levels and/or inhibit eIF4A in cytosol or in an individual in need of a decrease in activity of eIF4A, decrease in eIF4A activity and/or inhibition of eIF4A activity levels by administering an inhibiting amount of compound(s) of formulae (I), (I.a), (I.b), (A), (B) and an enantiomeric mixture as defined above or a salt thereof.

In one embodiment, the present invention provides a combined preparation of compound(s) of formulae (I), (I.a), (I.b), (A), (B) and an enantiomeric mixture as defined above, and/or a pharmaceutically acceptable salt thereof and/or prodrugs or isotope enriched forms thereof, and an additional therapeutic agent(s) for simultaneous, separate or sequential use in the treatment and/or prophylaxis of multiple diseases, preferably of proliferative disorders (e.g. cancer), in particular disorders associated with the activity of eIF4A.

Additional therapeutic agent(s) are selected from chemotherapeutic agents, radiotherapeutic agents, immuno-oncology agents, and combinations thereof.

In one aspect, the compound(s) of formulae (I), (I.a), (I.b), (A), (B) and an enantiomeric mixture as defined above are sequentially administered prior to administration of the immuno-oncology agent. In another aspect, compound(s) of formulae (I), (I.a), (I.b), (A), (B) and an enantiomeric mixture as defined above are administered concurrently with the immuno-oncology agent. In yet another aspect, compound(s) of formulae (I), (I.a), (I.b), (A), (B) and an enantiomeric mixture as defined above are sequentially administered after administration of the immuno-oncology agent.

In another aspect, compound(s) of formulae (I), (I.a), (I.b), (A), (B) and an enantiomeric mixture as defined above may be co-formulated with an immuno-oncology agent.

Immuno-oncology agents include, for example, a small molecule drug, antibody or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Suitable of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTpR, LIGHT, DcR3, HVEM, VEGETLIA, TRAMP/DR3, EDAR, EDA1, XEDAR, EDA2, TNFR1, Lymphotoxin a/TNFp, TNFR2, TNFa, LTpR, Lymphotoxin a 1b2, FAS, FASL, RELT, DR6, TROY, NGFR.

In one aspect, T cell responses can be stimulated by a combination of compound(s) of formulae (I), (I.a), (I.b), (A), (B) and an enantiomeric mixture as defined above and one or more of
(i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and
(ii) an agonist of a protein that stimulates T cell activation such as B7-1, B7-2, CD28, 4-1BB (CD 137), 4-1 BBL, ICOS, ICOS-L, 0X40, OX40L, GITR, GITRL, CD70, CD27, CD40, DR3 and CD28H.

Other agents that can be combined with compound(s) of formulae (I), (I.a), (I.b), (A), (B) and an enantiomeric mixture as defined above for the treatment of cancer include antagonists of inhibitory receptors on NK cells or agonists of activating receptors on NK cells. For example, compound(s) of formulae (I), (I.a), (I.b), (A), (B) and an enantiomeric mixture as defined above can be combined with antagonists of KIR, such as Lirilumab.

Yet other agents for combination therapies include agents that inhibit or deplete macrophages or monocytes, including but not limited to CSF-1 R antagonists such as CSF-1 R antagonist antibodies including RG7155.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy can also embrace the administration of the therapeutic agents as described above in further combination with other biologically active ingredients and non-drug therapies (e.g surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Types of cancers that may be treated with the compound of formulae (I), (I.a), (I.b), (A), (B) and an enantiomeric mixture as defined above include, but are not limited to, prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, haematological malignancies (including blood, bone marrow and lymph nodes) or testicular seminoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compound(s) of formulae (I), (I.a), (I.b), (A), (B) and an enantiomeric mixture as defined above for treatment of elF4A associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compound(s) of formulae (I), (I.a), (I.b), (A), (B) and an enantiomeric mixture as defined include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compound(s) of formulae (I), (I.a), (I.b), (A), (B) and an enantiomeric mixture as defined above may also be combined with immunotherapy drugs, including cytokines such as interferon alpha, interleukin 2, and tumor necrosis factor (TNF) in the treatment of melanoma. Compound(s) of formulae (I), (I.a), (I.b), (A), (B) and an enantiomeric mixture as defined above may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compound(s) of formulae (I), (I.a), (I.b), (A), (B) and an enantiomeric mixture as defined, using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-α), etoposide, and teniposide.

Other cytotoxic agents include navelbene, CPT-11, anastrazole, letrazole, capecitabine, reloxafme, and droloxafme.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1 BB and PD-1, or antibodies to cytokines (IL-IO or TGF-b).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

In a specific embodiment of the present invention, at least one compound of formulae (I), (I.a), (I.b), (A), (B) and an enantiomeric mixture as defined above and at least one chemotherapeutic agent are administered to the patient concurrently or sequentially. In other words, at least one compound(s) of formulae (I), (I.a), (I.b), (A), (B) and an enantiomeric mixture as defined above may be administered first, at least one chemotherapeutic agent may be administered first, or at least one compound of formulae (I), (I.a), (I.b), (A), (B) and an enantiomeric mixture as defined above may be administered at the same time. Additionally, when more than one compound of formulae (I), (I.a), (I.b), (A), (B) and an enantiomeric mixture as defined above and/or chemotherapeutic agent is used, the compounds may be administered in any order.

The invention also provides pharmaceutically compositions which comprise a therapeutically effective amount of one or more of the compound(s) of formulae (I), (I.a), (I.b), (A), (B) and an enantiomeric mixture as defined above, formulated together with one or more pharmaceutically acceptable carriers (additives) and/or diluents, and optionally, one or more additional therapeutic agents described above.

The compound(s) of formulae (I), (I.a), (I.b), (A), (B) and an enantiomeric mixture as defined above, may be administered by any suitable route, preferably in the form of a pharmaceutical composition adapted to such a route, and in a dose effective for the treatment intended. The compound(s) and compositions of the compound(s) of formulae (I), (I.a), (I.b), (A), (B) and an enantiomeric mixture as defined above, can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g. as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or animal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, bittering agents, coloring agents, demulcents, antioxidants, and preserving agents.

A tablet can, for example, be prepared by admixing at least one compound of formulae (I), (I.a), (I.b), (A), (B) and an enantiomeric mixture as defined above, and/or at least one pharmaceutically acceptable salt thereof and/or prodrugs or isotope enriched forms thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasantly tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of formulae (I), (I.a), (I.b), (A), (B) and an enantiomeric mixture as defined above, and/or at least one salt thereof and/or prodrugs or isotope enriched forms thereof with at least one inert solid diluent, such as, for example, calcium carbonate; calcium phosphate; and kaolin.

Soft gelatin capsules can, for example, be prepared by mixing at least one compound of formulae (I), (I.a), (I.b), (A), (B) and an enantiomeric mixture as defined above, and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formulae (I), (I.a), (I.b), (A), (B) and an enantiomeric mixture as defined above and/or at least one pharmaceutically acceptable salt and/or prodrugs or isotope enriched forms thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, hydroxypropyl-methylcellulose and hydroxypropyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formulae (I), (I.a), (I.b), (A), (B) and an enantiomeric mixture as defined above and/or at least one pharmaceutically acceptable salt thereof and/or prodrugs or isotope enriched forms thereof in either a vegetable oil, such as, for example, arachis oil, olive oil, sesame oil and coconut oil or in mineral oil, such as, for example, liquid paraffin. An oily suspension can also contain at least one thickening agent, such as, for example, beeswax, hard paraffin and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formulae (I), (I.a), (I.b), (A), (B) and an enantiomeric mixture as defined above and/or at least one pharmaceutically acceptable salt thereof and/or prodrugs or isotope enriched forms thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of formulae (I), (I.a), (I.b), (A), (B) and an enantiomeric mixture as defined above and/or at least one pharmaceutically acceptable salt thereof and/or prodrugs or isotope enriched forms thereof can, for example, be prepared as an oil-in-water emulsion. The oily phase of the emulsions comprising compound(s) of formulae (I), (I.a), (I.b), (A), (B) and an enantiomeric mixture as defined above may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilize) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compound(s) of formulae (I), (I.a), (I.b), (A), (B) and an enantiomeric mixture as defined above and/or at least one pharmaceutically acceptable salt thereof and/or prodrugs or isotope enriched forms thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, water or with cyclodextrin solubilization (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of formulae (I), (I.a), (I.b), (A), (B) and an enantiomeric mixture as defined above in an oily phase, such as, for example, a mixture of soybean oil and lecithin; 2) combining the compound(s) of formulae (I), (I.a), (I.b), (A), (B) and an enantiomeric mixture as defined above containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, AIIen, L. V. Jr. et al. Remington: The Science and Practice of Pharmacy (2 Volumes), 22nd Edition (2012), Pharmaceutical Press.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol poly ethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depend on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of formulae (I), (I.a), (I.b), (A), (B) and an enantiomeric mixture as defined above and/or at least one pharmaceutically acceptable salt thereof and/or prodrugs or isotope enriched forms thereof, and optionally an additional agent selected from any pharmaceutically acceptable carrier, adjuvant, and vehicle. Alternate compositions of this invention comprise a compound of the formulae (I), (I.a), (I.b), (A), (B) and an enantiomeric mixture as defined above, or a prodrug or isotope enriched forms thereof, and a pharmaceutically acceptable carrier, adjuvant, or vehicle.

The present invention also includes pharmaceutical kits useful, for example, in the treatment or prevention of elF4a-associated diseases. Thus, the present invention also relates to a kit containing a formulation comprising: a) a pharmaceutical composition comprising a compound of formulae (I), (I.a), (I.b), (A), (B) and an enantiomeric mixture as defined above, or a therapeutically acceptable salt thereof and/or prodrugs or isotope enriched forms thereof and a pharmaceutically acceptable carrier; and b) instructions for dosing of the pharmaceutical composition for the treatment of a disorder in which inhibition of the activity eIF4A, is effective in treating the disorder.

Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compound(s) of the formulae (I), (I.a), (I.b), (A), (B) and an enantiomeric mixture may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g. oral tablets, capsules, elixirs, and syrups, and consistent with conventional pharmaceutical practices. Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compound of the formulae (I), (I.a), (I.b), (A), (B) and an enantiomeric mixture (250 mg), lactose (75 mg), and magnesium stearate (15 mg). The mixture is passed through a 60 mesh sieve and packed into a no. 1 gelatin capsule.

A typical injectable preparation is produced by aseptically placing at least one of the compound of the formulae (I), (I.a), (I.b), (A), (B) and an enantiomeric mixture (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compound of formulae (I), (I.a), (I.b), (A), (B) and an enantiomeric mixture as defined above, alone or in combination with a pharmaceutical carrier. Optionally, compound(s) of formulae (I), (I.a), (I.b), (A), (B) and an enantiomeric mixture as defined above can be used alone, in combination with other compound(s) of formulae (I), (I.a), (I.b), (A), (B) and an enantiomeric mixture as defined above, or in combination with one or more other therapeutic agent(s), e.g. an anticancer agent or other pharmaceutically active material.

Regardless of the route of administration selected, the compound(s) of formulae (I), (I.a), (I.b), (A), (B) and an enantiomeric mixture as defined above, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound(s) of formulae (I), (I.a), (I.b), (A), (B) and an enantiomeric mixture as defined above employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of compound(s) of formulae (I), (I.a), (I.b), (A), (B) and an enantiomeric mixture as defined above employed in the pharmaceutical composition at levels lower than that required in order to achieve the desired therapeutic effect and gradually increase the dosage until the desired effect is achieved.

In general, a suitable daily dose of compound(s) of formulae (I), (I.a), (I.b), (A), (B) and an enantiomeric mixture as defined above will be that amount of the compound which is the lowest dose effective to produce a therapeutic effect. Such an effective dose will generally depend upon the factors described above. Generally, oral, intravenous, intracerebroventricular and subcutaneous doses of the compound(s) of formulae (I), (I.a), (I.b), (A), (B) and an enantiomeric mixture as defined above for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day, every other day, twice per week or per week.

While it is possible for compound(s) of formulae (I), (I.a), (I.b), (A), (B) and an enantiomeric mixture as defined above to be administered alone, it is preferable to administer the compound as a pharmaceutical formulation (composition).

The above other therapeutic agents, when employed in combination with the compound(s) of formulae (I), (I.a), (I.b), (A), (B) and an enantiomeric mixture as defined above, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

The invention will be illustrated further with reference to the examples that follow, without restricting the scope to the specific embodiments described. The invention includes all combinations of described and especially of preferred features that do not exclude each other.

### DESCRIPTION OF THE DRAWINGS

FIG. 1: MTT assay for cell viability in 96 well cell culture plate

Cells with RAS mutations were treated with compounds 1 and C-1 for 48 h and cell viability was evaluated by MTT assay. The results shown are mean from 3 independent experiments.

FIG. 2: The effect of the compounds in Ras activation.

NanoBiT assay for the RAS GTP-loading was performed in HeLa cells transfected with LgBit-K, N and HRAS G12V and SmBit-CRAF-RBD. Cells were treated with compounds 1 or C-1 for 2 h in serum-free DMEM (Dulbecco's modified Eagle's Medium). After incubation, the substrate for NanoLuc was added, and the luminescence was measured using a multiplate reader. Data were normalized to cells transfected with the indicated mutant and exposed to DMSO for 2 h. DMSO-treated cells were set as 1. The results shown are mean from 3 independent experiments.

FIG. 3: Dual luciferase assay for Cap-dependent translation initiation Dual luciferase assay for Cap-dependent translation initiation was performed in HeLa cells transfected with dual luciferase reporter gene based on pFR_HCV_xb. Cells were treated with compounds 1 or C-1 for 24 h in serum-free DMEM (Dulbecco's modified Eagle's Medium). After incubation, Dual luciferase reporter assay was performed according to the manufacturer's instruction and the luminescence was measured using a multiplate reader. Data were normalized to transfected cells treated with DMSO for 24 h. DMSO-treated cells were set as 1. The results shown are mean from 3 independent experiments.

FIG. 4: Western blot analysis of JEKO cells treated with compound 1.

JEKO cells (mantle cell lymphoma cell line) cells were treated with vehicle (DMSO) or compound 1 for various time points and the cell lysates were prepared. The proteins were separated by SDS-PAGE and the expression of C-myc and cyclin D1 was analysed by immunoblot analysis. Vinculin was used as a loading control.

FIG. 5: Structures of compounds 1, 2 and C-1.

### EXAMPLES

### Abbreviations

ACN acetonitrile
AIBN azo-bis-(isobutylonitrile)
DCE Dichloroethane
DMEM Dulbecco's modified Eagle's Medium
DCM dichloromethane
DIPEA Diisopropylethylamine
DMAP Dimethylaminopyridine
DMSO dimethylsulfoxid
FA Formic acid
FBS fetal bovine serum
IPA Isopropyl alcohol
LDA lithium diisopropylamide
NBS N-bromosuccinimide
PBS phosphate-buffered saline
PE Pet-ether
RLU relative light unit (luciferase activity)/relative luciferase units
SD standard deviation
SEM trimethylsilylethoxymethyl
TBAF tetra-n-butylammonium fluoride
TFA Trifluoroacetic acid
THF tetrahydrofuran
TLC thin-layer chromatography
TMSCN trimethylsilyl cyanide
Triton B benzyltrimethylammonium hydroxide

### Materials and Methods

### Cell:

HCT-116 were authenticated by Eurofin genomics and cultured in DMEM (10% heat inactivated FBS). sNF96.2 were purchased from ATCC and cultured in DMEM (10% heat inactivated FBS+1 mM Na Pyruvate). ASPC-1 cells were purchased from DSMZ and cultured in RPMI-1640 (10% heat inactivated FBS). NCI-H358 were purchased from ATCC and cultured in RPMI-1640 (10% heat inactivated FBS). T24 and HCT-116 cells were cultured in Mccoy's 5A medium (10% heat inactivated FBS). HT-1080 cells were cultured in EMEM (10% heat inactivated FBS+1 mM Na Pyruvate).

### DNA Transfection:

HeLa cells were harvested with 0.05% Trypsin/0.02% EDTA in PBS and seeded in 6 well or 12 well cell culture plates at the concentration of 5×10⁴ cells in complete DMEM (2 ml for 6 well plate and 1 ml for 12 well plate). After 1 day from seeding, the DNA were transfected with Flag tagged Ras or NanoBiT plasmid using PEI/PBS solution.

NanoBit assay.

The plasmids were transfected to HeLa cells and the cells were harvested after 1 day of transfection. The cells were seeded to 96 well white plate. Next day, the medium was changed to serum free DMEM for 2 h with compounds. After incubation, Nano Glo assay was performed. The luminescence was measured using Tecan SPARKS (Tecan). IC50 calculations by non-linear regression were conducted with GraphPad Prism 9.

### Dual Luciferase Assay:

Hela cells were seeded at 1×10⁶ cells/ml, 2 ml in 6 well cell culture plate in growth medium. 2 μg of plasmids were transfected into cells with 0.5 mM of PEI reagent in 200 μl PBS. One day after transfection, cells were harvested and seeded into 96 well white plates, half area (Greiner). After an additional day the medium was changed to serum free DMEM and the cells were incubated for 24 h with Compounds. Dual-Gbo Luciferase Assay was performed according to the manufacturer's instructions (Promega, N2920). The luminescence was measured using Tecan infinite (Tecan). IC50 calculations by non-linear regression were conducted with GraphPad Prism 9.

### Mtt Assay:

Cancer cells were seeded in 96 well plates (5×10³ cells/well) in 80 μl in growth medium and cultured for 1 day in the incubator. Next day, 20 μl of compounds containing growth medium was added to each well and the cells were cultured for 48 h. After incubation with compounds, 10 μl of MTT solution was added to the wells and incubated for 2-4 hours. After incubation with MTT, solubilization buffer was added and incubated over night. MTT was measured at O.D. 570 nm with a plate reader (Tecan). IC50 calculations by non-linear regression were conducted with GraphPad Prism 9.

### Synthesis

### Compound A: 4-((1R,3S,3aR,8bS)-3-(3-fluorophenyl)-1,8b-dihydroxy-6,8-dimethoxy-1,2,3,8b-tetrahydro-3aH-cyclopenta[b]benzofuran-3a-yl)benzonitrile

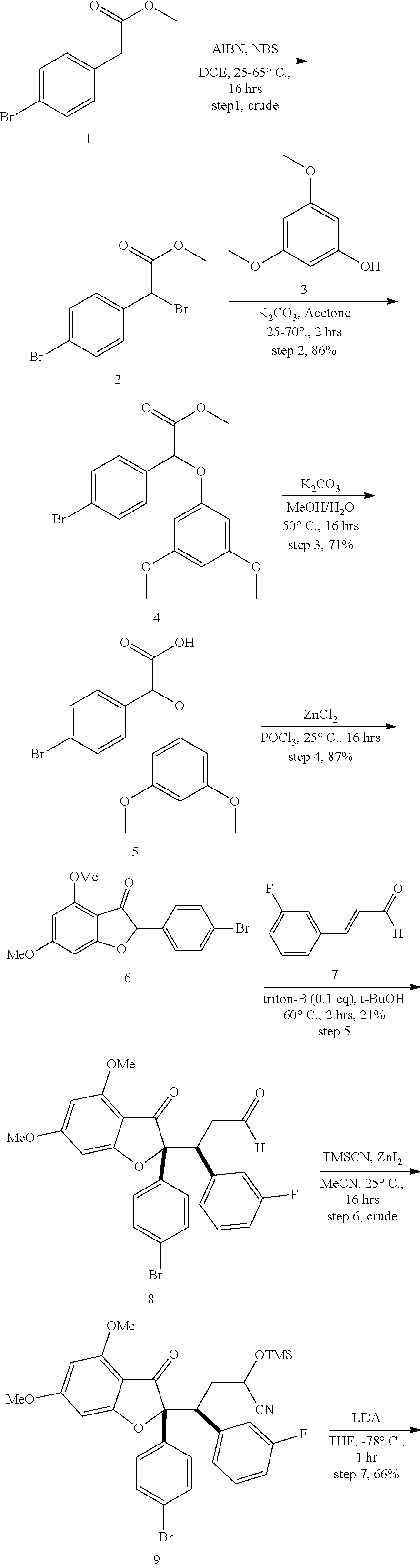

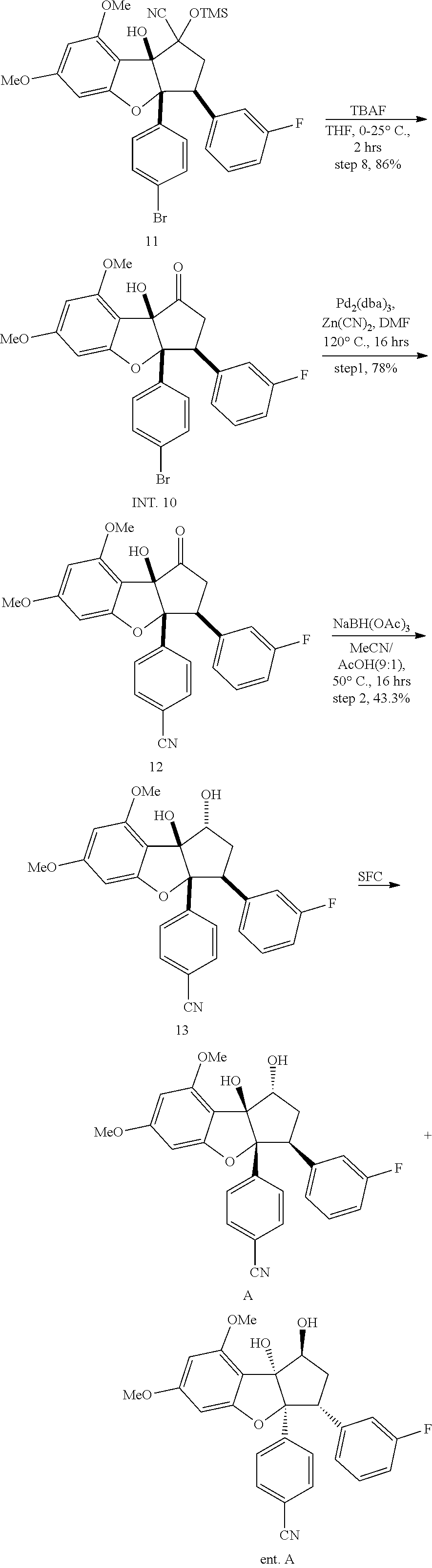

### Compound 2

To a solution of compound 1 (50 g, 219.3 mmol, 1 eq) in DCE (500 mL) was added AIBN (3.23 g, 19.64 mmol, 0.09 eq) and NBS (46.62 g, 261.93 mmol, 1.2 eq) at 25° C. Then the mixture was stirred at 65° C. under N₂ for 16 hrs. TLC (Petroleum ether/Ethyl acetate=10/1, R_{f}=0.8) showed compound 1 was consumed completely and one main peak with desired mass was detected. The reaction mixture was quenched by water (500 mL) at 25° C., extracted with DCM (1 L*2). The combined organic layers were washed with brine (500 mL), dried over Na₂SO₄, filtered and concentrated under reduced pressure to give to compound 2 (74 g, crude) as white solid which was used for the next step without further purification.

### Compound 4

To a solution of crude compound 2 (60 g, ~176.5 mmol, 90% purity, 1 eq) in acetone (420 mL) was added K₂CO₃ (41.20 g, 298.08 mmol, 1.7 eq) and 3, 5-dimethoxyphenol (30.00 g, 194.63 mmol, 1.11 eq) at 25° C. Then the mixture was stirred at 70° C. under N₂ for 16 hrs. TLC (Petroleum ether/Ethyl acetate=10/1, R_{f}=0.60) indicated compound 2 was consumed completely and one new spot formed. The reaction mixture was filtered and concentrated, diluted with H₂O, extracted with EtOAc three times. The combined organic layers were washed with brine (500 mL), dried over Na₂SO₄, filtered and concentrated under reduced pressure to give crude compound 4. The residue was purified by column (SiO₂, petroleum ether/ethyl acetate=100/1 to 5/1) to give compound 4 (57.6 g, 151.6 mmol, 86% yield over 2 steps) as a white solid. ¹H NMR (400 MHz, CDCl₃) 5=7.59-7.50 (m, 2H), 7.49-7.41 (m, 2H), 6.11 (s, 3H), 5.57 (s, 1H), 3.82-3.70 (m, 9H).

### Compound 5

To a solution of compound 4 (60 g, 157.9 mmol, 1 eq) in MeOH (420 mL) and H₂O (42 mL) was added K₂CO₃ (26.10 g, 188.87 mmol, 1.2 eq) at 25° C. The mixture was stirred at 50° C. under N₂ for 16 hrs. TLC (Petroleum ether/Ethyl acetate=1:1, R_{f}=0.60) indicated compound 4 was consumed completely and one new spot formed. Four parallel reactions were combined and worked up together. The reaction mixture was quenched by water (1 L) at 25° C., then the reaction was adjusted pH with HCl (6 mol/L), extracted with EtOAc (1 L*3). The combined organic layers were washed with brine (1 L), dried over Na₂SO₄, filtered and concentrated under reduced pressure to give a residue. Then the residue was triturated by Petroleum ether/Ethyl acetate=20:1, filtered and concentrated under reduced pressure to give compound 5 (325 g, 888.0 mmol, 71%) as a white solid. ¹H NMR (400 MHz, CDCl₃) 5=7.60-7.50 (m, 2H), 7.49-7.40 (m, 2H), 6.21-6.01 (m, 3H), 5.58 (s, 1H), 3.75 (s, 6H).

### Compound 6

To a mixture of compound 5 (25 g, 68.3 mmol, 1 eq) and ZnCl₂ (12.99 g, 95.32 mmol, 4.46 mL, 1.4 eq) was added POCl₃ (100 mL) at 25° C., then the mixture was stirred at 25° C. for 16 hrs. TLC (Petroleum ether/Ethyl acetate=2:1, R_{f}=0.50) indicated compound 5 was consumed completely and one new spot formed. Four parallel reactions were combined and worked up together. The reaction mixture was quenched by slowly dropping into water (1000 mL) at 25-40° C., filtered to give a residue. Then the residue was washed with water (3 times), filtered and concentrated under reduced pressure to give a crude product. Then the residue was triturated with methyl alcohol, filtered and concentrated under reduced pressure to give compound 6 (83 g, 238.50 mmol, 87% yield) as a white solid. ¹H NMR (400 MHz, CDCl₃) 5=7.53-7.48 (m, 2H), 7.31 (d, J=8.3 Hz, 2H), 6.27 (d, J=1.9 Hz, 1H), 6.06 (d, J=1.8 Hz, 1H), 5.43 (s, 1H), 3.91 (d, J=2.0 Hz, 6H).

### Compound 8

To a solution of compound 6 (10 g, 28.7 mmol, 1 eq) in t-BuOH (360 mL) was added triton B (1.20 g, 2.86 mmol, 1.30 mL, 40% purity, 0.1 eq) and the solution of compound 7 (5.16 g, 34.37 mmol, 1.2 eq) in t-BuOH (40 mL) at 25° C. The mixture was stirred at 60° C. under N₂ for 2 hrs. LCMS showed compound 6 was consumed completely and two peaks with desired mass was detected. The solvent was removed in vacuo, then the mixture was quenched by water (50 mL) and acidified with 6 M HCl to pH=1. The remained liquid was extracted with ethyl acetate (100 mL×2), dried over Na₂SO₄, filtered and concentrated under reduced pressure to give a residue. The residue was purified by column chromatography (SiO2, petroleum ether/ethyl acetate=10/1 to 5/1) to give compound 8 (3.01 g, 6.0 mmol, 21% yield) as a white solid and its diastereoisomer 8A (1.32 g, 2.65 mmol, 9% yield) as white solid. Compound 8: ¹H NMR (400 MHz, CDCl₃) 5=9.44 (d, J=1.1 Hz, 1H), 7.67-7.58 (m, 2H), 7.54-7.44 (m, 2H), 7.17-7.09 (m, 2H), 7.06-6.99 (m, 1H), 6.84-6.76 (m, 1H), 6.24 (d, J=1.9 Hz, 1H), 5.85 (d, J=1.8 Hz, 1H), 4.20 (dd, J=3.8, 10.8 Hz, 1H), 3.87 (s, 3H), 3.73 (s, 3H), 3.06 (ddd, J=2.1, 10.9, 17.7 Hz, 1H), 2.75-2.51 (m, 1H). Compound 8A: ¹H NMR (400 MHz, CDCl₃) 5=9.51 (d, J=2.1 Hz, 1H), 7.39-7.34 (m, 2H), 7.32-7.28 (m, 2H), 7.09 (dt, J=6.1, 7.9 Hz, 1H), 7.02-6.96 (m, 1H), 6.89 (d, J=7.9 Hz, 1H), 6.82 (dt, J=2.6, 8.3 Hz, 1H), 6.37 (d, J=1.8 Hz, 1H), 6.05 (d, J=1.8 Hz, 1H), 4.16 (dd, J=3.4, 11.1 Hz, 1H), 3.94 (s, 3H), 3.89 (s, 3H), 2.97 (ddd, J=2.5, 11.0, 17.2 Hz, 1H), 2.65 (dd, J=3.4, 17.3 Hz, 1H).

### Compound 9

To a solution of compound 8 (22.5 g, 45.2 mmol, 1 eq) in CH₃CN (225 mL) was added TMSCN (14.31 g, 144.19 mmol, 18.04 mL, 3.2 eq) and ZnI₂ (1.44 g, 4.51 mmol, 0.1 eq) at 25° C. The mixture was stirred at 25° C. under N₂for 16 hrs. LCMS showed compound 8 was consumed completely and one main peak with desired mass was detected. The reaction mixture was quenched by water (200 mL), extracted with EtOAc (150 mL×2). The combined organic layers were washed with brine (100 mL), dried over Na₂SO₄, filtered and concentrated under reduced pressure to give compound 9 (27.15 g, crude) as yellow solid. The residue was used to the next step without further purification. ¹H NMR (400 MHz, CDCl₃) δ=7.63-7.55 (m, 2H), 7.54-7.49 (m, 2H), 7.23-7.10 (m, 2H), 7.10-6.95 (m, 2H), 6.91-6.82 (m, 1H), 6.26 (t, J=2.2 Hz, 1H), 5.85 (dd, J=1.9, 3.0 Hz, 1H), 3.88 (s, 4H), 3.72 (d, J=1.8 Hz, 3H), 2.01 (s, 3H), 0.05 (d, J=3.8 Hz, 9H).

### Compound 11

To a solution of compound 9 (21.6 g, 36.2 mmol, 1 eq) in THE (432 mL) was added dropwise LDA (2 M, 54.13 mL, 3 eq) at −78° C. The mixture was stirred at −78° C. under N₂ for 1 hr. LCMS showed compound 9 was consumed completely and one main peak with desired mass was detected. The reaction mixture was quenched by H₂O/ice (500 mL), extracted with EtOAc (500 mL×2). The combined organic layers were washed with brine (500 mL), dried over Na₂SO₄, filtered and concentrated under reduced pressure to give a residue. The residue was purified by column chromatography (SiO2, petroleum ether/ethyl acetate=10/1 to 5/1) to give compound 11 (14.2 g, 23.7 mmol, 66% yield) as yellow solid and INT.10 (2.5 g, 5.0 mmol, 14% yield) as yellow solid. Compound 11 ¹H NMR (400 MHz, CDCl₃) δ=7.29 (br d, J=8.4 Hz, 1H), 7.20 (d, J=8.3 Hz, 2H), 7.11 (q, J=7.3 Hz, 1H), 7.01-6.94 (m, 2H), 6.85 (d, J=7.8 Hz, 1H), 6.82-6.73 (m, 2H), 6.21 (s, 1H), 6.05 (s, 1H), 4.32 (dd, J=5.6, 14.4 Hz, 1H), 3.85 (s, 3H), 3.82 (s, 3H), 3.17-2.99 (m, 2H), 2.68 (dd, J=5.7, 12.7 Hz, 1H), 0.11 (s, 9H).

### INT.10

To a solution of compound 11 (14.2 g, 23.8 mmol, 1 eq) in THE (142 mL) was added TBAF (1 M, 29.66 mL, 1.25 eq) at 0° C. The mixture was stirred at 25° C. under N2 for 2 hrs. LCMS showed compound 11 was consumed completely and one main peak with desired mass was detected. The reaction mixture was quenched by water (150 mL), extracted with EtOAc (150 mL×2). The combined organic layers were washed with brine (150 mL), dried over Na₂SO₄, filtered and concentrated under reduced pressure to give a residue. The residue was purified by column chromatography (SiO₂, petroleum ether/ethyl acetate=10/1 to 5/1) to give INT.10 (10.2 g, 20.5 mmol, 86% yield) as yellow solid. LCMS: M+23 (520.9), 92.7%. ¹H NMR (400 MHz, CDCl₃) δ=7.35-7.28 (m, 2H), 7.15-7.03 (m, 2H), 6.95 (d, J=8.6 Hz, 2H), 6.83 (br t, J=8.1 Hz, 1H), 6.74 (br d, J=8.1 Hz, 2H), 6.36 (d, J=1.8 Hz, 1H), 6.13 (d, J=1.7 Hz, 1H), 5.31 (s, 1H), 3.97-3.91 (m, 1H), 3.87 (s, 3H), 3.85 (s, 1H), 3.83 (s, 3H), 3.29 (br s, 1H), 3.13-3.02 (m, 1H), 3.02-2.90 (m, 1H).

### Compound 12

To a mixture of INT.10 (300 mg, 0.60 mmol, 1 eq) in DMF (6 mL) was added Zn(CN)₂ (282.19 mg, 2.40 mmol, 152.53 μL, 4 eq), Pd₂(dba)₃ (55.02 mg, 60.08 μmol, 0.1 eq) and DPPF (66.62 mg, 120.16 μmol, 0.2 eq) under N₂ at 20° C. The mixture was stirred at 120° C. for 16 hrs. LCMS showed INT.10 was consumed and desired mass as major. The reaction mixture was quenched by water (120 mL) at 25° C., extracted with EtOAc (100 mL×2). The combined organic layers were washed with brine (100 mL), dried over Na₂SO₄, filtered and concentrated under reduced pressure to give a residue. The residue was purified by prep-MPLC (SiO₂, Petroleum ether/Ethyl acetate=100/1 to 16/1) to give compound 12 (210 mg, 0.47 mmol, 78% yield) as a yellow solid. LCMS: M+1 (446.1), 80.1%. ¹H NMR (400 MHz, CDCl₃) δ=7.46 (d, J=8.4 Hz, 2H), 7.18 (d, J=8.4 Hz, 2H), 7.12-7.05 (m, 1H), 6.86-6.78 (m, 1H), 6.72 (br t, J=7.0 Hz, 2H), 6.37 (d, J=1.6 Hz, 1H), 6.14 (d, J=1.4 Hz, 1H), 4.01-3.92 (m, 1H), 3.87 (s, 3H), 3.84 (s, 3H), 3.19-3.08 (m, 1H), 3.01 (br d, J=12.3 Hz, 1H).

### Compound 13

To a solution of compound 12 (0.23 g, 516.35 μmol, 1 eq) in MeCN (4.5 mL) and AcOH (0.5 mL) was added NaBH(OAc)₃ (2.19 g, 10.33 mmol, 20 eq) at 25° C., then the mixture was stirred at 50° C. for 16 hrs. LCMS showed the starting material was consumed completely and one main peak with desired mass was detected. The reaction mixture was quenched by water (30 mL) at 25° C., extracted with EtOAc (30 mL×2). The combined organic layers were washed with brine (20 mL), dried over Na₂SO₄, filtered and concentrated under reduced pressure to give a residue. The residue was purified by prep-HPLC (column: Phenomenex luna C18 100*40 mm*3 um; mobile phase: [H₂O (0.1% TFA)-ACN]; gradient:30%-60% B over 8.0 min) to give compound 13 (0.1 g, 223.49 μmol, 43% yield) as a yellow solid. LCMS: M+1 (448.15), 70.5%. ¹H NMR (400 MHz, CDCl₃) δ=7.47-7.40 (m, 2H), 7.38-7.31 (m, 2H), 7.11-7.01 (m, 1H), 6.83-6.76 (m, 1H), 6.76-6.69 (m, 2H), 6.31 (d, J=1.8 Hz, 1H), 6.18 (d, J=1.8 Hz, 1H), 4.82 (d, J=5.8 Hz, 1H), 4.10-4.03 (m, 1H), 3.91 (s, 3H), 3.86 (s, 3H), 2.73 (dt, J=6.2, 13.9 Hz, 1H), 2.24 (dd, J=6.5, 13.6 Hz, 1H).

### Compound A

Compound 13 (0.1 g, 223.49 μmol, 1 eq) was purified by SFC (column: DAICEL CHIRALPAK AD (250 mm*30 mm, 10 um); mobile phase: [CO₂—IPA]; B %:45%, isocratic elution mode) to give two peaks.

Peak 1, compound A (20.7 mg, 46.3 μmol) as a white solid. LCMS: M+23 (470.1), 97.8%. ¹H NMR (400 MHz, CDCl₃) δ=7.48-7.40 (m, 2H), 7.39-7.32 (m, 2H), 7.12-7.02 (m, 1H), 6.84-6.68 (m, 3H), 6.32 (s, 1H), 6.18 (s, 1H), 4.83 (br d, J=6.1 Hz, 1H), 4.08 (br dd, J=6.4, 14.1 Hz, 1H), 3.92 (s, 3H), 3.87 (s, 3H), 3.12 (s, 1H), 2.73 (dt, J=6.1, 14.0 Hz, 1H), 2.25 (dd, J=6.3, 13.6 Hz, 1H), 1.82 (s, 1H)

Peak 2, enantiomer of compound A (21.7 mg, 48.50 μmol) as a white solid. LCMS: M+23 (470.1), 99%. ¹H NMR (400 MHz, CDCl₃) δ=7.44 (br d, J=8.4 Hz, 2H), 7.35 (br d, J=8.4 Hz, 2H), 7.07 (br d, J=6.4 Hz, 1H), 6.88-6.68 (m, 3H), 6.32 (d, J=1.0 Hz, 1H), 6.18 (s, 1H), 4.83 (br d, J=5.8 Hz, 1H), 4.08 (br dd, J=6.6, 13.9 Hz, 1H), 3.92 (s, 3H), 3.87 (s, 3H), 3.12 (s, 1H), 2.73 (dt, J=6.1, 13.7 Hz, 1H), 2.25 (br dd, J=6.8, 13.9 Hz, 1H), 1.82 (s, 1H).

### Compound B: 4-((1R,3S,3aR,8bS)-1,8b-dihydroxy-6,8-dimethoxy-3-phenyl-1,2,3,8b-tetrahydro-3aH-cyclopenta[b]benzofuran-3a-yl)benzonitrile

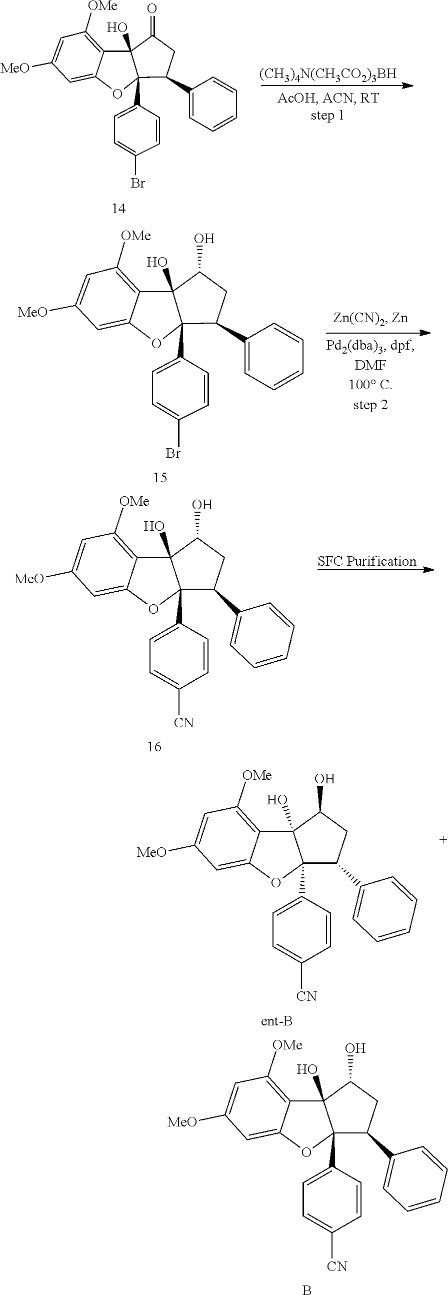

### Compound 15

To a solution of (CH₃)₄N(CH₃CO₂)₃BH (273 mg, 1.04 mmol) in acetonitrile (2 mL), AcOH (1 mL) was added at 0° C. and stirred 30 min at RT. Then a solution of (3S,3aR,8bR)-3a-(4-bromophenyl)-8b-hydroxy-6,8-dimethoxy-3-phenyl-2,3,3a,8b-tetrahydro-1H-cyclopenta[b]benzofuran-1-one (14) (50 mg, 0.10 mmol) in acetonitrile (2 mL) was added dropwise at 0° C. and the reaction mixture was stirred 7 h at RT. After completion of reaction (monitored by TLC), the reaction mixture was diluted with dilute HCl (1.5 N, 4 mL) at 0° C. and the aqueous layer was extracted with DCM (2×30 mL). The combined organic layer was washed with brine solution (4 mL), was dried over anhydrous Na₂SO₄. The organic part was filtered, concentrated under reduced pressure and the resulting crude compound was purified by flash column chromatography (silica gel: 230-400 mesh, eluent: 10-20% EtOAc/Petroleum ether as gradient) to afford compound 15 (40 mg, 80%) as a yellow solid. ¹H NMR (400 MHz, DMSO-d₆): δ 7.21 (d, J=11.6 Hz, 2H), 7.12-7.09 (m, 2H), 7.05-7.02 (m, 5H), 6.27 (d, J=2.0 Hz, 1H), 6.12 (d, J=2.0 Hz, 1H), 4.98 (s, 1H), 4.50-4.49 (m, 1H), 4.47-4.45 (m, 1H), 3.80 (dd, J=5.6, 14.0 Hz, 1H), 3.78 (s, 3H), 3.74 (s, 3H), 2.71-2.67 (m, 1H), 2.02 (dd, J=6.4, 12.8 Hz, 1H).

### Compound 16

To a solution of (1R,3S,3aR,8bS)-3a-(4-bromophenyl)-6,8-dimethoxy-3-phenyl-1,2,3,3a-tetrahydro-8bH-cyclopenta[b]benzofuran-1,8b-diol (35 mg, 0.07 mmol) in DMF (2 mL), Zinc cyanide (50.8 mg, 0.43 mmol) followed by zinc powder (0.47 mg, 7.24 μmol) were added at RT. The reaction mixture was degassed for 5 min by purging N₂ gas and dppf (8.03 mg, 0.01 mmol) followed by Pd₂(dba)₃ (6.63 mg, 7.24 μmol) were added at RT and the reaction mixture was stirred 2 h at 100° C. After completion of reaction (monitored by TLC), the reaction mixture was filtered through celite pad and washed with EtOAc (10 mL) followed by water (4 mL). The combined organic layer was extracted with EtOAc (20 mL), the organic part was washed with brine solution (5 mL) and was dried over anhydrous Na₂SO₄. The organic part was filtered and concentrated under vacuum, the resulting crude was purified by preparative HPLC to afford compound 16 (12 mg, 39%) as an off white solid). LCMS: 412.2 (M⁺-OH), ¹H-NMR (400 MHz, DMSO-d₆): δ 7.50 (d, J=8.4 Hz, 2H), 7.27 (d, J=8.4 Hz, 2H), 7.12-7.08 (m, 2H), 7.04-7.00 (m, 3H), 6.29 (d, J=2.0 Hz, 1H), 6.13 (d, J=2.0 Hz, 1H), 5.10 (s, 1H), 4.58 (d, J=3.6 Hz, 1H), 4.47-4.45 (m, 1H), 4.08 (dd, J=6.4, 14.8 Hz, 1H), 3.78 (s, 3H), 3.74 (s, 3H), 2.77-2.76 (m, 1H), 2.09 (dd, J=5.6, 12.8 Hz, 1H).

### Compound B

Compound 16 (20 mg, 0.047 mmol) was separated by chiral SFC purification (CO₂:Methanol 60:40 to 80:20, flow rate: 3 mL/min; column: YMC Cellulose-SJ). The first eluting peak was concentrated under vacuum at 45° C. to afford ent-B and the second eluting peak afforded compound B.

Enantiomer of compound B: 9 mg, off white solid). LCMS: 412.2 (M⁺-OH). ¹H-NMR (400 MHz, DMSO-d₆): δ 7.50 (d, J=8.4 Hz, 2H), 7.27 (d, J=8.4 Hz, 2H), 7.11-7.08 (m, 2H), 7.04-7.00 (m, 3H), 6.29 (d, J=2.0 Hz, 1H), 6.13 (d, J=2.0 Hz, 1H), 5.10 (s, 1H), 4.58 (d, J=3.6 Hz, 1H), 4.47-4.45 (m, 1H), 4.08 (dd, J=6.4, 14.8 Hz, 1H), 3.78 (s, 3H), 3.74 (s, 3H), 2.81-2.73 (m, 1H), 2.07 (dd, J=5.6, 12.8 Hz, 1H).

Compound B: 6 mg, off white solid). LCMS: 430.0 (M++H). ¹H-NMR (400 MHz, DMSO-d₆): δ 7.50 (d, J=8.4 Hz, 2H), 7.27 (d, J=8.4 Hz, 2H), 7.12-7.08 (m, 2H), 7.04-7.02 (m, 3H), 6.29 (d, J=2.0 Hz, 1H), 6.13 (d, J=2.0 Hz, 1H), 5.10 (s, 1H), 4.58 (d, J=3.6 Hz, 1H), 4.47-4.45 (m, 1H), 4.08 (dd, J=6.4, 14.8 Hz, 1H), 3.78 (s, 3H), 3.74 (s, 3H), 2.77-2.74 (m, 1H), 2.08 (dd, J=5.6, 12.8 Hz, 1H).

### Compound C-1 (1R,3S,3aR,8bS)-3a-(4-chlorophenyl)-6,8-dimethoxy-3-phenyl-1,2,3,3a-tetrahydro-8bH-cyclopenta[b]benzofuran-1,8b-diol

(N. Ribeiro et al., J. Med. Chem., 2012, 55, 100064) was made in an analogous way.

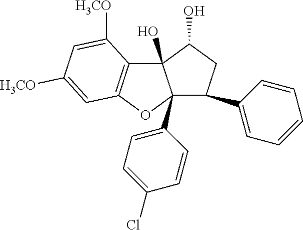 [SMILES: COc1cc(OC)c2c(c1)O[C@@]1(c3ccc(Cl)cc3)[C@H](c3ccccc3)C[C@@H](O)[C@@]21O]

LCMS: 439.2 (M++H). ¹H NMR (400 MHz, DMSO-d₆): δ 7.10-7.08 (m, 6H), 7.03-7.01 (m, 3H), 6.26 (d, J=2.0 Hz, 1H), 6.13 (d, J=2.0 Hz, 1H), 4.98 (s, 1H), 4.49-4.48 (m, 1H), 4.47-4.46 (m, 1H), 4.00 (dd, J=5.6, 6.4 Hz, 1H), 3.78 (s, 3H), 3.75 (s, 3H), 2.75-2.70 (m, 1H), 2.05-2.00 (m, 1H).

### Results:

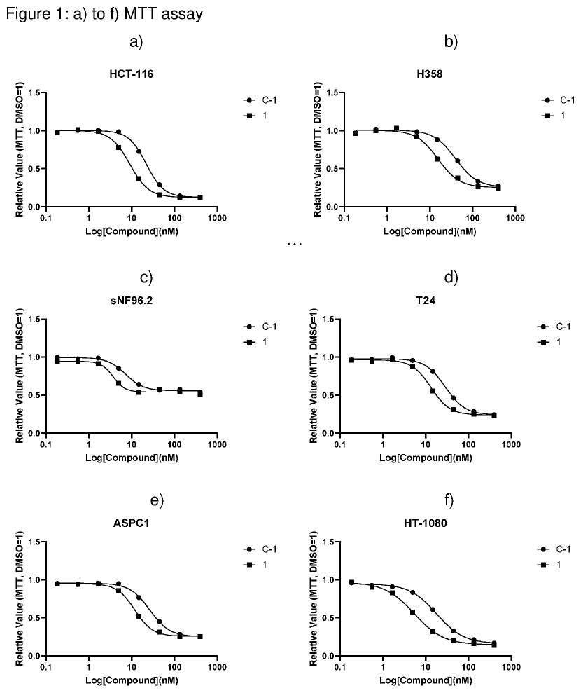

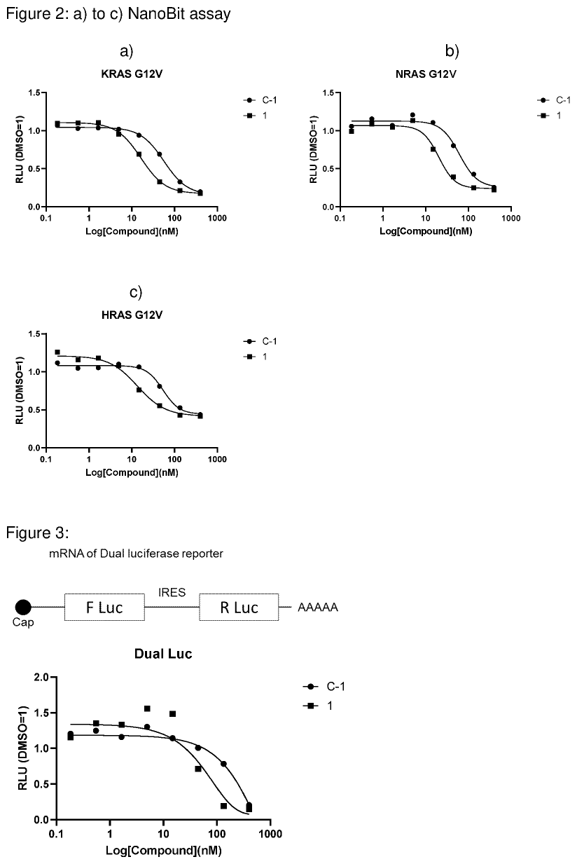

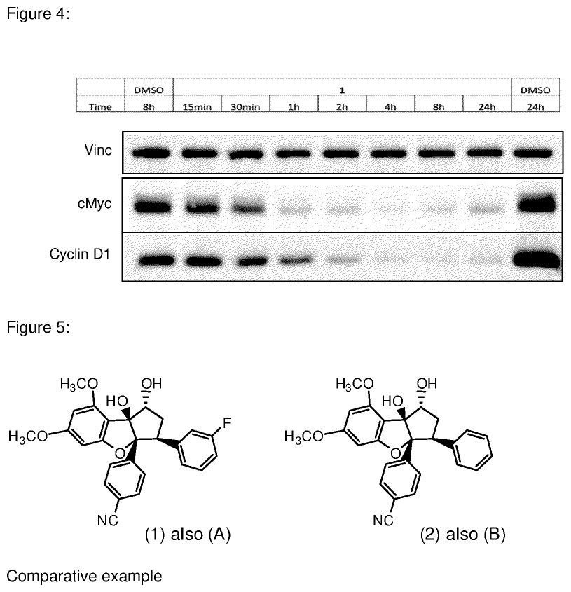

### FIG. 1a):

| HCT-116 | C-1 | 1 |
|---|---|---|
| IC50 (nM) | 21.87 | 8.99 |

| H358 | C-1 | 1 |
|---|---|---|
| IC50 (nM) | 40.00 | 15.58 |

| sNF96.2 | C-1 | 1 |
|---|---|---|
| IC50 (nM) | 7.22 | 3.69 |

| T24 | C-1 | 1 |
|---|---|---|
| IC50 (nM) | 28.48 | 13.25 |

| ASPC1 | C-1 | 1 |
|---|---|---|
| IC50 (nM) | 26.58 | 11.88 |

| HT-1080 | C-1 | 1 |
|---|---|---|
| IC50 (nM) | 18.02 | 5.39 |

| KRAS G12V | C-1 | 1 |
|---|---|---|
| IC50 (nM) | 56.58 | 16.46 |

| NRAS G12V | C-1 | 1 |
|---|---|---|
| IC50 (nM) | 61.39 | 20.22 |

| HRAS G12V | C-1 | 1 |
|---|---|---|
| IC50 (nM) | 53.49 | 13.56 |

| Dual luc | C-1 | 1 |
|---|---|---|
| IC50 (nM) | 389.20 | 57.08 |

### Unbound Fraction in Human Plasma and Pharmacokinetics (DMPK) (Table 1 Table 3)

After administration, a certain amount of drug is in the blood or plasma. Of the total amount in the blood or plasma, some is bound to proteins (or other species in the blood or plasma). Only unbound drug is available e.g., for drug-target interactions. Therefore, the higher the unbound fraction that is available in the blood or plasma, the more is available for binding the drug to a target. The less unbound drug is available in the blood or plasma e.g., because it is involved in competitive binding to, for example, albumin, the less is available for desired drug-target interaction. The protocol for determining the unbound fraction and pharmacokinetics is routine practice and known to the skilled person.

Surprisingly the compounds according to the invention have a much higher free fraction in human plasma. There is much more drug available for binding to a target compared to the compounds of the prior art.

The human microsomal clearance (hMic Clint) is lower for compound according to the invention compared to C-1. Since the exposure (AUC, area under the concentration-time curve) of a drug depends, inter alia, on its clearance (the lower the clearance of a drug, the higher the exposure). Typically, the beneficial effect of a drug depends on its potency and exposure, so a lower clearance corresponds to a lower dose to provide the same beneficial effect.

AUC=(F·D)/CL

where F is bioavailability (=100% for iv dosing), D is dose and CL is clearance.

TABLE 1
The iv (intravenous) PK of the three relevant compounds
at 2 mg/kg iv is compared. The conclusion is that the
free AUC and Cave is higher for 1 and 2 than for C-1.

| | C-1 (2 mg/ | 1 (2 mg/ | 2 (2.0 mg/ |
|---|---|---|---|
| | kg, i.v.) | kg, i.v.) | kg, i.v.) |
| Cmax (μM) | 1.7 | 1.2 | 1.7 |
| Cmax (nM), Free | 14.5 | 30.4 | 43.7 |
| AUCinf(h*μM) | 0.9 | 0.8 | 2.1 |
| AUCinf(h*nM) Free | 7.9 | 19.8 | 51.8 |
| T½ (h) | 1.3 | 0.9 | 4.0 |
| IC50 (nM, KRAS G12V) | 29.0 | 8.5 | 5.4 |
| Unbound fraction (%) | 0.9 | 2.6 | 2.5 |
| C average (nM) free | 0.3 | 0.8 | 2.2 |

TABLE 2
The oral PK of C-1 and 1 at 50 mg/kg po is compared. The conclusion
is that the free AUC and Cave is higher for 1 than for C-1.

| | C-1 (50 mg/kg, PO) | 1 (50 mg/kg, PO) |
|---|---|---|
| Cmax (μM) | 3.9 | 4.1 |
| Cmax (nM), Free | 34.3 | 105.3 |
| AUCinf(h*μM) | 12.6 | 14.6 |
| AUCinf(h*nM) Free | 109.8 | 378.6 |
| T½ (h) | 2.7 | 3.5 |
| IC50 (nM, KRAS G12V) | 29.0 | 8.5 |
| Unbound fraction (%) | 0.9 | 2.6 |
| C average (nM) free | 4.6 | 15.8 |

TABLE 3
The oral PK of C-1 and 2 at 10 mg/kg po is compared. The conclusion
is that the free AUC and Cave is higher for 2 than for C-1.

| | C-1 (10 mg/kg, PO) | 2 (10 mg/kg, PO) |
|---|---|---|
| Cmax (μM) | 0.3 | 0.9 |
| Cmax (nM), Free | 2.3 | 21.4 |
| AUCinf(h*μM) | 0.6 | 1.1 |
| AUCinf(h*nM) Free | 4.9 | 26.6 |
| T½ (h) | 1.7 | 1.3 |
| IC50 (nM, KRAS G12V) | 29.0 | 5.4 |
| Unbound fraction (%) | 0.9 | 2.5 |
| C average (nM) free | 0.2 | 1.1 |

TABLE 4
This table summarizes the unbound clearance data. The
conclusion is that the unbound AUC's of 1 and 2
are higher because the unbound clearance is lower.

| | C-1 | 1 | 2 |
|---|---|---|---|
| Cmax (nM), Free | 14.5 | 30.4 | 43.7 |
| Unbound clearance | 9689.7 | 3683.3 | 1580.9 |
| (ml/min/kg) | | | |
| AUCinf(h*nM) Free | 7.9 | 19.8 | 51.8 |
| T½ (h) | 1.3 | 0.9 | 4.0 |
| IC50 (nM, KRAS G12V) | 29.0 | 8.5 | 5.4 |
| Unbound fraction (%) | 0.9 | 2.6 | 2.5 |

### Protocol for Unbound Fraction

### Dialysis Setup:

HT-Dialysis plates (Model HTD 96 b, Cat #1006) and the dialysis membrane (molecular weight cut off 12-14 KDa, Cat #1101) were purchased from HT Dialysis LLC (Gales Ferry, CT).

Dialysis Buffer (100 mM sodium phosphate and 150 mM NaCl, pH 7.4±0.1)

Stop Solution (Acetonitrile containing tolbutamide at 200 ng/mL, labetalol at 200 ng/mL)

### Processing Procedure of Dialysis Membrane and Matrix:

On the day of experiment, the plasma was thawed under running cold tap water and centrifuged at 3220×g for 5 min to remove any clots. The pH value was checked and recorded. Only plasma within a range of pH 7.0 to pH 8.0 was used.

The dialysis membrane was pretreated according to the manufacturer's instructions: the dialysis membrane strips were soaked in ultra-pure water at room temperature for approximately 1 hr. Afterwards, each membrane strip that contains 2 membranes was separated and soaked in ethanol:water (20:80 v:v) for approximately 20 min, after which it was ready for use or was stored in the solution at 2-8° C. for up to 1 month. Prior to the experiment, the membrane was rinsed and soaked for 20 min in ultra-pure water for use.

### Dilution Procedure of Test Compound and Control Compound:

Working solutions (400 μM) of test compound and control compound were prepared.

Aliquots of working solutions (3 μL) were spiked into blank matrix (597 μL) to achieve final concentrations and mixed thoroughly.

### Procedure of Assay

Aliquots of 50 μL loading matrix containing test compound or control compound in triplicate was transferred to Sample Collection Plate, respectively. The samples were matched with opposite blank buffer to obtain a final volume of 100 μL with a volume ratio of matrix:Dialysis Buffer (1:1, v:v) in each well immediately. The stop solution was added to these TO samples of test compound and control compound. The plate was sealed and shaken at 800 rpm for 10 min. Then these TO samples were stored at 2-8° C. pending further process along with other post-dialysis samples.

The dialysis instrument was assembled following manufacturer's instructions.

An aliquot of 100 μL of the loading matrix containing test compound or control compound was transferred to the donor side of each dialysis well in triplicate and 100 μL of the Dialysis Buffer was loaded to the receiver side of the well. Then the plate was rotated at approximately 100 rpm in a humidified incubator with 5% CO₂ at 37±1° C. for 4 hours.

At the end of the dialysis, aliquots of 50 μL samples from the buffer side and matrix side of the dialysis device were taken into new 96-well plates (Sample Collection Plates). An equal volume of opposite blank matrix (buffer or matrix) in each sample was added to reach a final volume of 100 μL with volume ratio of matrix: dialysis buffer at 1:1 (v:v) in each well. AII samples were further processed by protein precipitation for LC-MS/MS analysis. The detailed processing method of samples were described in the study record.

### Data Analysis:

The % Unbound, % Bound and % Recovery were calculated using the following equations:

$$\% Unbound = 100 \times F / T$$

$$\% Bound = 100 - \% Unbound$$

$$\% Recovery = 100 \times ( F + T ) / T 0$$

F=Free compound concentration as determined by the calculated concentration on the buffer side of the membrane
T=Total compound concentration as determined by the calculated concentration on the matrix side of the membrane
T0=Total compound concentration as determined by the calculated concentration in matrix before dialysis

1. Compounds of formula (I) 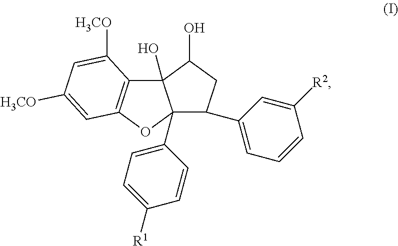
prodrugs or isotope enriched forms thereof or a pharmaceutically acceptable salt thereof,
wherein
R¹ is CN,
R² is selected from hydrogen and F.

2. The compounds according to claim 1 selected from compounds of formula (I.a) or (I.b) or an enantiomeric mixture comprising the compounds of formula (I.a) and (I.b) or prodrugs or isotope enriched forms thereof or the pharmaceutically acceptable salts thereof 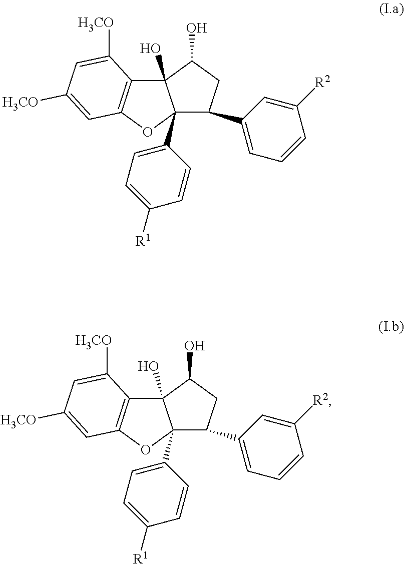
wherein R¹ and R² have the same meanings as defined in claim 1.

3. The compounds of formula (I) according to claim 1, being a mixture of (I.a) and (I.b), prodrugs or isotope enriched forms thereof or a pharmaceutically acceptable salts thereof, wherein the enantiomer excess (ee) of the enantiomer of formula (I.a) is at least 20% optionally being a mixture of (I.a) and (I.b), prodrugs or isotope enriched forms thereof or a pharmaceutically acceptable salts thereof, wherein the enantiomer excess (ee) of the enantiomer of formula (I.a) is at least 50%, optionally being a mixture of (I.a) and (I.b), prodrugs or isotope enriched forms thereof or a pharmaceutically acceptable salts thereof, wherein the enantiomer excess (ee) of the enantiomer of formula (I.a) is at least 80%, optionally being a mixture of (I.a) and (I.b), prodrugs or isotope enriched forms thereof or a pharmaceutically acceptable salts thereof, wherein the enantiomer excess (ee) of the enantiomer of formula (I.a) is at least 99%.

4. (canceled)

5. (canceled)

6. (canceled)

7. The compounds of formulae (I) or (I.a) or (I.b) or an enantiomeric mixture comprising the compounds of formula (I.a) and (I.b) or prodrugs or isotope enriched forms thereof or a pharmaceutically acceptable salt thereof, according to claim 1, wherein R² is hydrogen.

8. The compounds of formulae (I) or (I.a) or (I.b) or an enantiomeric mixture comprising the compounds of formula (I.a) and (I.b) or prodrugs or isotope enriched forms thereof or a pharmaceutically acceptable salt thereof, according to claim 1, wherein R² is F.

9. The compounds according to claim 1, selected from compounds of the formulae A and B and the mixture of each of compound A to B with its respective enantiomer,

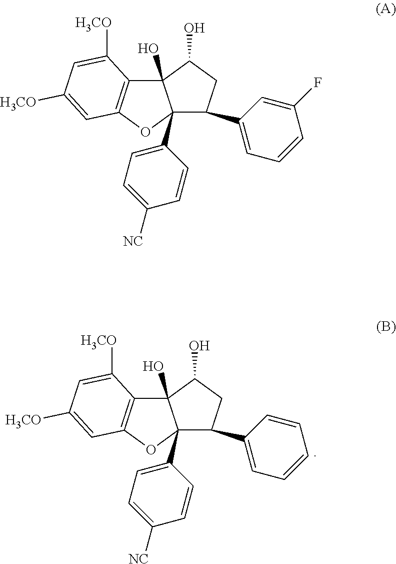 [SMILES: COc1cc(OC)c2c(c1)O[C@@]1(c3ccc(C#N)cc3)[C@H](c3cccc(F)c3)C[C@@H](O)[C@@]21O ; COc1cc(OC)c2c(c1)O[C@@]1(c3ccc(C#N)cc3)[C@H](c3ccccc3)C[C@@H](O)[C@@]21O]

10. Compounds of formulae (I), (I.a), (I.b), an enantiomeric mixture comprising the compounds of formula (I.a) and (I.b) or prodrugs or isotope enriched forms thereof or a pharmaceutically acceptable salt thereof, as defined in anyone of claims 1 to 9, for use as a medicament, optionally for use in the treatment and/or Prophylaxis of diseases, optionally for use in the treatment and/or prophylaxis of proliferative disorders or genetic disorders.

11. (canceled)

12. (canceled)

13. The compounds of formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined in claim 1, for use in the treatment and/or prophylaxis of proliferative disorders or genetic disorders which involves oncogenic RAS or eIF4A complex that controls the expression of genes selected from c-myc, cyclin D1.

14. The compounds of formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined in claim 1, for use in the treatment and/or prophylaxis of a disease, wherein the disease is selected from neoproliferative diseases, cancers, RASOpathies and inflammatory disorders, in particular CFC (Cranio Facial Cutaneous), NF1 (Neurofibromatosis Type 1).

15. The compounds formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined in claim 1, for use as inhibitor of RAS protein activation, or for use as inhibitor or ligand of eIF4A complex or for use as a ligand of Prohibitin, or use in treating proliferative disorders wherein RAS-signaling is involved, or use in treating proliferative disorders wherein eIF4A-Myc-signalinq is involved.

16. (canceled)

17. (canceled)

18. (canceled)

19. The compounds formulae (I) or (I.a) or (I.b) or an enantiomeric mixture comprising the compounds of formula (I.a) and (I.b) or prodrugs or isotope enriched forms thereof or a pharmaceutically acceptable salt thereof, as defined in claim 1, for use in treating proliferative disorders, wherein KRAS G12V, NRAS G12V, HRAS G12V, KRAS G12C, KRAS G12D, KRAS G12C/Y96D, KRAS G13C, KRAS G13D, KRASG13S, KRAS Q61H, KRAS Q61R or KRAS Q61 K is involved, or use in treatinq proliferative disorders wherein c-myc is overexpressed and/or wherein cyclin D1 is overexpressed, or use in treatinq proliferative disorders wherein any activatinq mutation in KRAS, HRAS and NRAS is involved, or use in treatinq proliferative disorders wherein any mutation that acquires resistance to RAS inhibitors is involved or use in treatinq proliferative disorders, wherein any mutation in EGFR or its related genes that contributes to acquired resistance to RAS inhibitors.

20. (canceled)

21. (canceled)

22. (canceled)

23. (canceled)

24. A method of inhibiting growth, proliferation, or metastasis of cancer cells in a subject in need thereof, said method comprising administering to the subject a therapeutically effective amount of at least one compound selected from compounds of formulae (I), (I.a), (I.b), an enantiomeric mixture comprising the compounds of formula (I.a) and (I.b) or prodrugs or isotope enriched forms thereof or a pharmaceutically acceptable salt thereof as defined in claim 1.

25. A pharmaceutical composition, comprising a pharmaceutically effective amount of at least one compound selected from compounds of formulae (I), (I.a), (I.b), an enantiomeric mixture comprising the compounds of formula (I.a) and (I.b) or prodrugs or isotope enriched forms thereof or a pharmaceutically acceptable salt thereof, as defined in claim 1, and one or more pharmaceutically acceptable carriers.

26. The pharmaceutical composition according to claim 25, for use in the prophylaxis and/or treatment of proliferative disorders or genetic disorders.

27. A kit containing a formulation comprising: a1) at least one compound selected from compounds of formulae (I), (I.a), (I.b), an enantiomeric mixture comprising the compounds of formula (I.a) and (I.b) or prodrugs or isotope enriched forms thereof or a pharmaceutically acceptable salt thereof, as defined in claim 1, or a2) a pharmaceutical composition according to claim 25; and b) instructions for dosing of the pharmaceutical composition for the treatment of a disorder in which inhibition of RAS activation or the downstream signaling pathways, in particular C-Myc upregulation and/cyclin D1 upregulation is effective in treating the disorder.

28. A method of treatment and/or prophylaxis of a disease or condition mediated by RAS, eIF4A, or a prohibitin (PHB/2) in a subject in need thereof, comprising administering to the subject the compound of claim 1,
wherein said disease or condition is the result of any mutation in RAS in said subject that leads to reduced activity of a RAS inhibitor, or
wherein said disease or condition is the result of dysregulation of eIF4A in said subject, or
wherein said disease or condition is the result of overexpression of one or more prohibitins in said subject.
